SEQUENCE LISTING (126 sequences)

SEQ ID NO: 1 - protein, 43 aa, synthetic construct
  VARIANT 1: note = X is DTyr, His, Phe, Tyr or absent
  VARIANT 3: note = X is Asp, Gln, Glu or His
  VARIANT 12: note = X is Ile or Lys
  VARIANT 17: note = X is Gln or Ile
  VARIANT 18: note = X is Ala, Arg or His
  VARIANT 19: note = X is Ala or Gln
  VARIANT 2: note = X is AIB, Ala, or absent
  VARIANT 7: note = X is Ile, Thr or Ser
  VARIANT 10: note = X is Tyr or His
  VARIANT 13: note = X is AIB, Ala, Gln, Glu, Gly, His, Pro, Ser, Thr, Tyr, Val, Asp or Asn
  VARIANT 14: note = X is Leu, Met or NLeu
  VARIANT 20: note = X is AIB, Gln, His or Lys
  VARIANT 21: note = X is Ala, Asp or Glu
  VARIANT 24: note = X is Asn, Gln or Glu
  VARIANT 25: note = X is Arg, His, Trp or Tyr
  VARIANT 27: note = X is Ile or Leu
  VARIANT 28: note = X is Ala or Asn
  VARIANT 29: note = X is Gln, Gly or Thr
  VARIANT 30: note = X is Gly, Lys, or Lys substituted at its epsilon amino group with a group of the formula Z-Xaa44-Xaa45-Xaa46-Xaa47-, or absent
  VARIANT 31: note = X is Arg, Gly, Pro, or absent
  VARIANT 32: note = X is Asn, Lys, or Lys substituted at its epsilon amino group with a group of the formula Z-Xaa44-Xaa45-Xaa46-Xaa47-, or absent
  VARIANT 33: note = X is Lys, Lys substituted at its epsilon amino group with a group of the formula Z-Xaa44-Xaa45-Xaa46-Xaa47-, or absent
  VARIANT 34: note = X is Asn, or Lys substituted at its epsilon amino group with a group of the formula Z-Xaa44-Xaa45-Xaa46-Xaa47-, or absent
  VARIANT 35: note = X is Asp or absent
  VARIANT 36: note = X is Trp, or Lys substituted at its epsilon amino group with a group of the formula Z-Xaa44-Xaa45-Xaa46-Xaa47-, or absent
  VARIANT 37: note = X is Lys, or Lys substituted at its epsilon amino group with a group of the formula Z-Xaa44-Xaa45-Xaa46-Xaa47-, or absent
  VARIANT 38: note = X is His or absent
  VARIANT 39: note = X is Asn or Lys substituted at its epsilon amino group with a group of the formula Z-Xaa44-Xaa45-Xaa46-Xaa47-, or absent
  VARIANT 40: note = X is Ile, Leu, Lys substituted at its epsilon amino group with a group of the formula Z-Xaa44-Xaa45-Xaa46-Xaa47-, or absent
  VARIANT 41: note = X is Thr, or absent
  VARIANT 42: note = X is Gln, or absent
  VARIANT 43: note = X is Lys substituted at its epsilon amino group with a group of the formula Z-Xaa44-Xaa45-Xaa46-Xaa47-, or absent
XXXGTFXSDXSXXXDKXXXXXFVXXLXXXXXXXXXXXXXXXXX

SEQ ID NO: 2 - protein, 42 aa, Homo sapiens
YAEGTFISDYSIAMDKIHQQDFVNWLLAQKGKKNDWKHNITQ

SEQ ID NO: 3 - protein, 31 aa, Homo sapiens
HAEGTFTSDVSSYLEGQAAKEFIAWLVKGRG

SEQ ID NO: 4 - protein, 30 aa, Homo sapiens
  MOD_RES 30: note = The C-terminal amino acid has a -C(O)NH2 group in place of a carboxylic acid group
HAEGTFTSDVSSYLEGQAAKEFIAWLVKGR

SEQ ID NO: 5 - protein, 29 aa, Homo sapiens
HSQGTFTSDYSKYLDSRRAQDFVQWLMNT

SEQ ID NO: 6 - protein, 37 aa, Homo sapiens
HSQGTFTSDYSKYLDSRRAQDFVQWLMNTKRNRNNIA

SEQ ID NO: 7 - protein, 29 aa, synthetic construct
  VARIANT 1: note = X is DTyr, His, Phe or Tyr, or absent
  VARIANT 2: note = X is AIB, Ala, or absent
  VARIANT 3: note = X is Asp, Gln, Glu or His
  VARIANT 7: note = X is Ile, Thr or Ser
  VARIANT 10: note = X is Tyr or His
  VARIANT 12: note = X is Ile or Lys
  VARIANT 13: note = X is AIB, Ala, Gln, Glu, Gly, His, Pro, Ser, Thr, Tyr, Val, Asp or Asn
  VARIANT 14: note = X is Leu, Met, or NLeu
  VARIANT 17: note = X is Gln or Ile
  VARIANT 18: note = X is Ala, Arg or His
  VARIANT 19: note = X is Ala or Gln
  VARIANT 20: note = X is AIB, Gln, His or Lys
  VARIANT 21: note = X is Ala, Asp, or Glu
  VARIANT 24: note = X is Asn, Gln or Glu
  VARIANT 25: note = X is Arg, His, Trp or Tyr
  VARIANT 27: note = X is Ile or Leu
  VARIANT 28: note = X is Ala or Asn
  VARIANT 29: note = X is Gln, Gly or Thr
XXXGTFXSDXSXXXDKXXXXXFVXXLXXX

SEQ ID NO: 8 - protein, 14 aa, synthetic construct
  VARIANT 1: note = X is Gly, Lys or Lys substituted at its epsilon amino group with a group of the formula Z-Xaa44-Xaa45-Xaa46-Xaa47-
  VARIANT 2: note = X is Gly or Pro
  VARIANT 3: note = X is Lys or Lys substituted at its epsilon amino group with a group of the formula Z-Xaa44-Xaa45-Xaa46-Xaa47-
  VARIANT 4: note = X is Lys or Lys substituted at its epsilon amino group with a group of the formula Z-Xaa44-Xaa45-Xaa46-Xaa47-
  VARIANT 7: note = X is Trp or Lys substituted at its epsilon amino group with a group of the formula Z-Xaa44-Xaa45-Xaa46-Xaa47-
  VARIANT 8: note = X is Lys or Lys substituted at its epsilon amino group with a group of the formula Z-Xaa44-Xaa45-Xaa46-Xaa47-
  VARIANT 10: note = X is Asn or Lys substituted at its epsilon amino group with a group of the formula Z-Xaa44-Xaa45-Xaa46-Xaa47-
  VARIANT 11: note = X is Ile, Leu or Lys substituted at its epsilon amino group with a group of the formula Z-Xaa44-Xaa45-Xaa46-Xaa47-
  VARIANT 14: note = X is Lys substituted at its epsilon amino group with a group of the formula Z-Xaa44-Xaa45-Xaa46-Xaa47- or absent
XXXXNDXXHXXTQX

SEQ ID NO: 9 - protein, 29 aa, synthetic construct
  VARIANT 1: note = X is DTyr, His, Phe, Tyr, or absent
  VARIANT 2: note = X is AIB, Ala, or absent
  VARIANT 3: note = X is Asp, Gln, Glu or His
  VARIANT 7: note = X is Ile or Thr
  VARIANT 13: note = X is AIB, Ala, Gln, Glu, Gly, His, Pro, Ser, Thr, Tyr or Val
  VARIANT 14: note = X is Leu, Met or NLeu
  VARIANT 17: note = X is Gln or Ile
  VARIANT 18: note = X is Ala, Arg, or His
  VARIANT 19: note = X is Ala or Gln
  VARIANT 20: note = X is AIB, Gln, His or Lys
  VARIANT 21: note = X is Ala, Asp or Glu
  VARIANT 25: note = X is Arg, His, Trp or Tyr
  VARIANT 24: note = X is Asn, Gln or Glu
  VARIANT 27: note = X is Ile or Leu
  VARIANT 28: note = X is Ala or Asn
  VARIANT 29: note = X is Gln, Gly or Thr
XXXGTFXSDYSIXXDKXXXXXFVXXLXXX

SEQ ID NO: 10 - protein, 5 aa, synthetic construct
DDDDY

SEQ ID NO: 11 - protein, 5 aa, synthetic construct
HPFHL

SEQ ID NO: 12 - protein, 43 aa, synthetic construct
  MOD_RES 43: note = Lys is substituted at its epsilon amino group with a group of the formula Z-Xaa44-Xaa45-Xaa46-Xaa47-
YAEGTFISDYSIAMDKIHQQDFVNWLLAQKGKKNDWKHNITQK

SEQ ID NO: 13 - protein, 43 aa, synthetic construct
  SITE 2: note = AIB
  MOD_RES 43: note = Lys is substituted at its epsilon amino group with a group of the formula Z-Xaa44-Xaa45-Xaa46-Xaa47-
YXEGTFISDYSIAMDKIHQQDFVNWLLAQKGKKNDWKHNITQK

SEQ ID NO: 14 - protein, 43 aa, synthetic construct
  SITE 2: note = AIB
  MOD_RES 43: note = Lys is substituted at its epsilon amino group with a group of the formula Z-Xaa44-Xaa45-Xaa46-Xaa47-
YXEGTFISDYSIALDKIHQQDFVNWLLAQKGKKNDWKHNITQK

SEQ ID NO: 15 - protein, 42 aa, synthetic construct
  SITE 2: note = AIB
  MOD_RES 30: note = Lys is substituted at its epsilon amino group with a group of the formula Z-Xaa44-Xaa45-Xaa46-Xaa47-
YXEGTFISDYSIALDKIHQQDFVNWLLAQKGKKNDWKHNITQ

SEQ ID NO: 16 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 17 - protein, 42 aa, synthetic construct; identical to SEQ ID NO: 15
  SITE 2: note = AIB
  MOD_RES 33: note = Lys is substituted at its epsilon amino group with a group of the formula Z-Xaa44-Xaa45-Xaa46-Xaa47-

SEQ ID NO: 18 - protein, 42 aa, synthetic construct
  SITE 2: note = AIB
  MOD_RES 36: note = Lys is substituted at its epsilon amino group with a group of the formula Z-Xaa44-Xaa45-Xaa46-Xaa47-
YXEGTFISDYSIALDKIHQQDFVNWLLAQKGKKNDKKHNITQ

SEQ ID NO: 19 - protein, 42 aa, synthetic construct; identical to SEQ ID NO: 15
  SITE 2: note = AIB
  MOD_RES 37: note = Lys is substituted at its epsilon amino group with a group of the formula Z-Xaa44-Xaa45-Xaa46-Xaa47-

SEQ ID NO: 20 - protein, 42 aa, synthetic construct
  SITE 2: note = AIB
  MOD_RES 39: note = Lys is substituted at its epsilon amino group with a group of the formula Z-Xaa44-Xaa45-Xaa46-Xaa47-
YXEGTFISDYSIALDKIHQQDFVNWLLAQKGKKNDWKHKITQ

SEQ ID NO: 21 - protein, 42 aa, synthetic construct
  SITE 2: note = AIB
  MOD_RES 40: note = Lys is substituted at its epsilon amino group with a group of the formula Z-Xaa44-Xaa45-Xaa46-Xaa47-
YXEGTFISDYSIALDKIHQQDFVNWLLAQKGKKNDWKHNKTQ

SEQ ID NO: 22 - protein, 30 aa, synthetic construct
  SITE 2: note = AIB
  MOD_RES 30: note = Lys is substituted at its epsilon amino group with a group of the formula Z-Xaa44-Xaa45-Xaa46-Xaa47-
YXEGTFISDYSIALDKIHQQDFVNWLLAQK

SEQ ID NO: 23 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQQDFVNWLLAQKGK

SEQ ID NO: 24 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSIALDKIHQQDFVNWLLAQKGKK

SEQ ID NO: 25 - protein, 41 aa, synthetic construct
  MOD_RES 41: note = Lys is substituted at its epsilon amino group with a group of the formula Z-Xaa44-Xaa45-Xaa46-Xaa47-
EGTFISDYSIAMDKIRQQDFVNWLLAQKGKKNDWKHNLTQK

SEQ ID NO: 26 - protein, 43 aa, synthetic construct; identical to SEQ ID NO: 13
  SITE 2: note = AIB
  MOD_RES 43: note = Lys is substituted at its epsilon amino group with a group of the formula Z-Xaa44-Xaa45-Xaa46-Xaa47-

SEQ ID NO: 27 - protein, 43 aa, synthetic construct; identical to SEQ ID NO: 13
  SITE 2: note = AIB
  MOD_RES 43: note = Lys is substituted at its epsilon amino group with a group of the formula Z-Xaa44-Xaa45-Xaa46-Xaa47-

SEQ ID NO: 28 - protein, 43 aa, synthetic construct; identical to SEQ ID NO: 13
  SITE 2: note = AIB
  MOD_RES 43: note = Lys is substituted at its epsilon amino group with a group of the formula Z-Xaa44-Xaa45-Xaa46-Xaa47-

SEQ ID NO: 29 - protein, 43 aa, synthetic construct; identical to SEQ ID NO: 13
  SITE 2: note = AIB
  MOD_RES 43: note = Lys is substituted at its epsilon amino group with a group of the formula Z-Xaa44-Xaa45-Xaa46-Xaa47-

SEQ ID NO: 30 - protein, 43 aa, synthetic construct; identical to SEQ ID NO: 13
  SITE 2: note = AIB
  MOD_RES 43: note = Lys is substituted at its epsilon amino group with a group of the formula Z-Xaa44-Xaa45-Xaa46-Xaa47-

SEQ ID NO: 31 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIAMDKIHQQDFVNWLLAQKGKKNDWKHNITQ

SEQ ID NO: 32 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
  SITE 14: note = Nle
YXEGTFISDYSIAXDKIHQQDFVNWLLAQKGKKNDWKHNITQ

SEQ ID NO: 33 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 34 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 35 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 36 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 37 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 38 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 39 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQHDFVNWLLAQKGKKNDWKHNITQ

SEQ ID NO: 40 - protein, 35 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQQDFVNWLLAQKGKKND

SEQ ID NO: 41 - protein, 38 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQQDFVNWLLAQKGKKNDWKH

SEQ ID NO: 42 - protein, 42 aa, synthetic construct
  SITE 2: note = AIB
  SITE 13: note = AIB
  MOD_RES 32: note = Lys is substituted at its epsilon amino group with a group of the formula Z-Xaa44-Xaa45-Xaa46-Xaa47-
YXEGTFISDYSIXLDKIHQQDFVNWLLAQKGKKNDWKHNITQ

SEQ ID NO: 43 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIGLDKIHQQDFVNWLLAQKGKKNDWKHNITQ

SEQ ID NO: 44 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIVLDKIHQQDFVNWLLAQKGXKNDWKHNITQ

SEQ ID NO: 45 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSISLDKIHQQDFVNWLLAQKGKKNDWKHNITQ

SEQ ID NO: 46 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSITLDKIHQQDFVNWLLAQKGKKNDWKHNITQ

SEQ ID NO: 47 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIPLDKIHQQDFVNWLLAQKGKKNDWKHNITQ

SEQ ID NO: 48 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIELDKIHQQDFVNWLLAQKGKKNDWKHNITQ

SEQ ID NO: 49 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 50 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 51 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 52 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 53 - protein, 42 aa, synthetic construct; identical to SEQ ID NO: 42
  SITE 2: note = AIB
  SITE 13: note = AIB
  MOD_RES 32: note = Lys is substituted at its epsilon amino group with a group of the formula Z-Xaa44-Xaa45-Xaa46-Xaa47-

SEQ ID NO: 54 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 55 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 56 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 57 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 58 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 59 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIHLDKIHQQDFVNWLLAQKGKKNDWKHNITQ

SEQ ID NO: 60 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIYLDKIHQQDFVNWLLAQKGKKNDWKHNITQ

SEQ ID NO: 61 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIQLDKIHQQDFVNWLLAQKGKKNDWKHNITQ

SEQ ID NO: 62 - protein, 43 aa, synthetic construct; identical to SEQ ID NO: 14
  SITE 2: note = AIB
  MOD_RES 43: note = Lys is substituted at its epsilon amino group with a group of the formula Z-Xaa44-Xaa45-Xaa46-Xaa47-

SEQ ID NO: 63 - protein, 43 aa, synthetic construct; identical to SEQ ID NO: 14
  SITE 2: note = AIB
  MOD_RES 43: note = Lys is substituted at its epsilon amino group with a group of the formula Z-Xaa44-Xaa45-Xaa46-Xaa47-

SEQ ID NO: 64 - protein, 32 aa, synthetic construct; identical to SEQ ID NO: 23
  SITE 2: note = AIB
  MOD_RES 32: note = Lys is substituted at its epsilon amino group with a group of the formula Z-Xaa44-Xaa45-Xaa46-Xaa47-

SEQ ID NO: 65 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSIALDKIHQQDFVNWLLAQKGKX

SEQ ID NO: 66 - protein, 32 aa, synthetic construct; identical to SEQ ID NO: 23
  SITE 2: note = AIB
  MOD_RES 32: note = Lys is substituted at its epsilon amino group with a group of the formula Z-Xaa44-Xaa45-Xaa46-Xaa47-

SEQ ID NO: 67 - protein, 32 aa, synthetic construct; identical to SEQ ID NO: 23
  SITE 2: note = AIB
  MOD_RES 32: note = Lys is substituted at its epsilon amino group with a group of the formula Z-Xaa44-Xaa45-Xaa46-Xaa47-

SEQ ID NO: 68 - protein, 33 aa, synthetic construct; identical to SEQ ID NO: 24
  SITE 2: note = AIB
  MOD_RES 33: note = Lys is substituted at its epsilon amino group with a group of the formula Z-Xaa44-Xaa45-Xaa46-Xaa47-

SEQ ID NO: 69 - protein, 34 aa, synthetic construct
  SITE 2: note = AIB
  MOD_RES 34: note = Lys is substituted at its epsilon amino group with a group of the formula Z-Xaa44-Xaa45-Xaa46-Xaa47-
YXEGTFISDYSIALDKIHQQDFVNWLLAQKGKKK

SEQ ID NO: 70 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXHGTFISDYSIALDKIHQQDFVNWLLAQKGKKNDWKHNITQ

SEQ ID NO: 71 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXQGTFISDYSIALDKIHQQDFVNWLLAQKGKKNDWKHNITQ

SEQ ID NO: 72 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
HXEGTFISDYSIALDKIHQQDFVNWLLAQKGKKNDWKHNITQ

SEQ ID NO: 73 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
HXHGTFISDYSIALDKIHQQDFVNWLLAQKGKKNDWKHNITQ

SEQ ID NO: 74 - protein, 42 aa, synthetic construct; identical to SEQ ID NO: 71
  SITE 2: note = AIB
  SITE 1: note = D-tyrosine
  MOD_RES 32: note = Lys is substituted at its epsilon amino group with a group of the formula Z-Xaa44-Xaa45-Xaa46-Xaa47-

SEQ ID NO: 75 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIALDKIHQQDFVNWLLAQKGKKNDWKHNITQ

SEQ ID NO: 76 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIAQQDFVNWLLAQKGKKNDWKHNITQ

SEQ ID NO: 77 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQKAFVNWLLAQKGKKNDWKHNITQ

SEQ ID NO: 78 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIALDKIAQKAFVQWLIAQKGKKNDWKHNITQ

SEQ ID NO: 79 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIALDKIAQKAFVQWLIAGGPKKNDWKHNITQ

SEQ ID NO: 80 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIALDKIAQKAFVQWLIAGGPK

SEQ ID NO: 81 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
  SITE 20: note = AIB
YXEGTFTSDYSIYLDKQAAXEFVNWLLAGGPK

SEQ ID NO: 82 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIYLDKQAQQDFVNWLLAGGPK

SEQ ID NO: 83 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFTSDYSIYLDKQAQQDFVNWLLNTKRNK

SEQ ID NO: 84 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFTSDYSIYLDKQAQQDFVNWLLAQKGK

SEQ ID NO: 85 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFTSDYSIYLDKQAQQDFVEWLLNTKRNK

SEQ ID NO: 86 - protein, 32 aa, synthetic construct; identical to SEQ ID NO: 23
  SITE 2: note = AIB
  MOD_RES 32: note = Lys is substituted at its epsilon amino group with a group of the formula Z-Xaa44-Xaa45-Xaa46-Xaa47-

SEQ ID NO: 87 - protein, 32 aa, synthetic construct
  SITE 2: note = AIB
  MOD_RES 32: note = Lys is substituted at its epsilon amino group with a group of the formula Z-Xaa44-Xaa45-Xaa46-Xaa47-
YXEGTFISDYSIALDKIHQQDFVEWLLAQKGK

SEQ ID NO: 88 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 89 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
FXEGTFISDYSIALDKIHQQDFVNWLLAQKGKKNDWKHNITQ

SEQ ID NO: 90 - protein, 42 aa, synthetic construct; identical to SEQ ID NO: 89
  SITE 2: note = AIB
  MOD_RES 32: note = Lys is substituted at its epsilon amino group with a group of the formula Z-Xaa44-Xaa45-Xaa46-Xaa47-

SEQ ID NO: 91 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
FXQGTFISDYSIALDKIHQQDFVNWLLAQKGKKNDWKHNITQ

SEQ ID NO: 92 - protein, 42 aa, synthetic construct
  SITE 2: note = AIB
  MOD_RES 32: note = Lys is substituted at its epsilon amino group with a group of the formula Z-Xaa44-Xaa45-Xaa46-Xaa47-
YXDGTFISDYSIALDKIHQQDFVNWLLAQKGKKNDWKHNITQ

SEQ ID NO: 93 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 94 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 95 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 96 - protein, 33 aa, synthetic construct; identical to SEQ ID NO: 24
  SITE 2: note = AIB
  MOD_RES 33: note = Lys is substituted at its epsilon amino group with a group of the formula Z-Xaa44-Xaa45-Xaa46-Xaa47-

SEQ ID NO: 97 - protein, 33 aa, synthetic construct; identical to SEQ ID NO: 24
  SITE 2: note = AIB
  MOD_RES 33: note = Lys is substituted at its epsilon amino group with a group of the formula Z-Xaa44-Xaa45-Xaa46-Xaa47-

SEQ ID NO: 98 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 99 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 100 - protein, 42 aa, synthetic construct; identical to SEQ ID NO: 15
  SITE 2: note = AIB
  MOD_RES 32: note = Lys is substituted at its epsilon amino group with a group of the formula Z-Xaa44-Xaa45-Xaa46-Xaa47-

SEQ ID NO: 101 - protein, 34 aa, synthetic construct; identical to SEQ ID NO: 69
  SITE 2: note = AIB
  MOD_RES 34: note = Lys is substituted at its epsilon amino group with a group of the formula Z-Xaa44-Xaa45-Xaa46-Xaa47-

SEQ ID NO: 102 - protein, 42 aa, synthetic construct; identical to SEQ ID NO: 42
  SITE 2: note = AIB
  SITE 13: note = AIB
  MOD_RES 32: note = Lys is substituted at its epsilon amino group with a group of the formula Z-Xaa44-Xaa45-Xaa46-Xaa47-

SEQ ID NO: 103 - protein, 42 aa, synthetic construct
  SITE 2: note = AIB
  SITE 13: note = AIB
  MOD_RES 32: note = Lys is substituted at its epsilon amino group with a group of the formula Z-Xaa44-Xaa45-Xaa46-Xaa47-
YXEGTFTSDYSIXLDKIHQQDFVNWLLAQKGKKNDWKHNITQ

SEQ ID NO: 104 - protein, 42 aa, synthetic construct; identical to SEQ ID NO: 15
  SITE 2: note = AIB
  MOD_RES 33: note = Lys is substituted at its epsilon amino group with a group of the formula Z-Xaa44-Xaa45-Xaa46-Xaa47-

SEQ ID NO: 105 - protein, 42 aa, synthetic construct; identical to SEQ ID NO: 15
  SITE 2: note = AIB
  MOD_RES 37: note = Lys is substituted at its epsilon amino group with a group of the formula Z-Xaa44-Xaa45-Xaa46-Xaa47-

SEQ ID NO: 106 - protein, 33 aa, synthetic construct; identical to SEQ ID NO: 24
  SITE 2: note = AIB
  MOD_RES 33: note = Lys is substituted at its epsilon amino group with a group of the formula Z-Xaa44-Xaa45-Xaa46-Xaa47-

SEQ ID NO: 107 - protein, 37 aa, synthetic construct
  SITE 2: note = AIB
  MOD_RES 37: note = Lys is substituted at its epsilon amino group with a group of the formula Z-Xaa44-Xaa45-Xaa46-Xaa47-
YXEGTFISDYSIALDKIHQQDFVNWLLAQKGKKNDWK

SEQ ID NO: 108 - protein, 33 aa, synthetic construct
  SITE 2: note = AIB
  SITE 13: note = AIB
  MOD_RES 33: note = Lys is substituted at its epsilon amino group with a group of the formula Z-Xaa44-Xaa45-Xaa46-Xaa47-
YXEGTFISDYSIXLDKIHQQDFVNWLLAQKGKK

SEQ ID NO: 109 - protein, 37 aa, synthetic construct
  SITE 2: note = AIB
  SITE 13: note = AIB
  MOD_RES 37: note = Lys is substituted at its epsilon amino group with a group of the formula Z-Xaa44-Xaa45-Xaa46-Xaa47-
YXEGTFISDYSIXLDKIHQQDFVNWLLAQKGKKNDWK

SEQ ID NO: 110 - protein, 33 aa, synthetic construct
  SITE 2: note = AIB
  SITE 13: note = AIB
  MOD_RES 33: note = Lys is substituted at its epsilon amino group with a group of the formula Z-Xaa44-Xaa45-Xaa46-Xaa47-
YXEGTFISDYSIXLDKIHQQDFVNHLLAQKGKK

SEQ ID NO: 111 - protein, 33 aa, synthetic construct
  SITE 2: note = AIB
  SITE 13: note = AIB
  MOD_RES 33: note = Lys is substituted at its epsilon amino group with a group of the formula Z-Xaa44-Xaa45-Xaa46-Xaa47-
YXEGTFISDYSIXLDKIHQQDFVNRLLAQKGKK

SEQ ID NO: 112 - protein, 33 aa, synthetic construct
  SITE 2: note = AIB
  SITE 13: note = AIB
  MOD_RES 33: note = Lys is substituted at its epsilon amino group with a group of the formula Z-Xaa44-Xaa45-Xaa46-Xaa47-
YXEGTFISDYSIXLDKIHQQDFVNYLLAQKGKK

SEQ ID NO: 113 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSIDLDKIHQQDFVNWLLAQKGKK

SEQ ID NO: 114 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSINLDKIHQQDFVNWLLAQKGKK

SEQ ID NO: 115 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSKDLDKIHQQDFVNWLLAQKGKK

SEQ ID NO: 116 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFSSDYSIALDKIHQQDFVNWLLAQKGKK

SEQ ID NO: 117 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSISLDKIHQQDFVNWLLAQKGKK

SEQ ID NO: 118 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDHSIALDKIHQQDFVNWLLAQKGKK

SEQ ID NO: 119 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFISDYSKALDKIHQQDFVNWLLAQKGKK

SEQ ID NO: 120 - protein, 34 aa, synthetic construct; identical to SEQ ID NO: 69
  SITE 2: note = AIB
  MOD_RES 34: note = Lys is substituted at its epsilon amino group with a group of the formula Z-Xaa44-Xaa45-Xaa46-Xaa47-

SEQ ID NO: 121 - protein, 33 aa, synthetic construct
  SITE 2: note = AIB
  MOD_RES 33: note = Lys is substituted at its epsilon amino group with a group of the formula Z-Xaa44-Xaa45-Xaa46-Xaa47-
YXEGTFSSDYSKDLDKIHQQDFVNWLLAQKGKK

SEQ ID NO: 122 - protein, 4 aa, synthetic construct
GSGS

SEQ ID NO: 123 - protein, 4 aa, synthetic construct
TGSG

SEQ ID NO: 124 - protein, 4 aa, synthetic construct
TGSG

SEQ ID NO: 125 - protein, 4 aa, synthetic construct
SGSG

SEQ ID NO: 126 - protein, 4 aa, synthetic construct
TGSG

(19) United States
(12) Patent Application Publication
(10) Pub. No.: US 2026/0242421 A1
(43) Pub. Date: Aug. 20, 2026

(54) NOVEL COMPOUNDS

(71) Applicant: IP2IPO Innovations Limited, London (GB)

(72) Inventor: Stephen Robert BLOOM, London (GB)

(21) Appl. No.: 19/159,280

(22) PCT Filed: Feb. 23, 2024
(86) PCT No.: PCT/GB2024/050504

§ 371 Date: Aug. 22, 2025

(30) Foreign Application Priority Data
Feb. 24, 2023 (GB) 2302686.7

(51) Int. Cl. C07K 14/00; A61K 38/00; A61P 3/04

(52) U.S. Cl. CPC C07K 14/001; A61P 3/04; A61K 38/00

(57) ABSTRACT

The invention provides novel compounds which are peptide hormone analogues, and which are useful in treating disorders such as diabetes and obesity. The compounds of the general sequence recited in the specification possess a tailored profile with regards to potency properties at the GIP receptor. With regard to in vivo properties, administration of example peptides of the invention have been shown, in animal models, to result in increased weight loss. Preferred compounds achieve this without reducing food intake significantly.

### FIELD

The present disclosure relates to compounds which are peptide hormone analogues, and which are useful in treating disorders such as diabetes and obesity.

### BACKGROUND

According to the National Health and Nutrition Examination Survey (NHANES, 2009-2010), 33.0% of adults in the United States aged 20 and over are overweight, 35.7% are obese, and 6.3% are extremely obese. In addition, a large percentage of children in the United States are overweight or obese.

The cause of obesity is complex and multi-factorial. Increasing evidence suggests that obesity is not a simple problem of self-control but is a complex disorder involving appetite regulation and energy metabolism. In addition, obesity is associated with a variety of conditions associated with increased morbidity and mortality in a population.

Although the etiology of obesity is not definitively established, genetic, metabolic, biochemical, cultural and psychosocial factors are believed to contribute. In general, obesity has been described as a condition in which excess body fat puts an individual at a health risk.

There is strong evidence that obesity is associated with increased morbidity and mortality. Disease risk, such as cardiovascular disease risk and type 2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (see Kenchaiah et al., N. Engl. J. Med. 347:305, 2002; Massie, N. Engl. J. Med. 347:358, 2002).

Diabetes is a chronic syndrome of impaired carbohydrate, protein, and fat metabolism owing to insufficient secretion of insulin or to target tissue insulin resistance. It occurs in two major forms: insulin-dependent diabetes mellitus (type 1 diabetes) and non-insulin dependent diabetes mellitus (type 2 diabetes). Diabetes type I, or insulin dependent diabetes mellitus (IDDM) is caused by the destruction of β cells, which results in insufficient levels of endogenous insulin. Diabetes type 2, or non-insulin dependent diabetes, results from a defect in both the body's sensitivity to insulin, and a relative deficiency in insulin production. According to the National Diabetes Statistics Report, 2014 around 28.9 million adults in the United States aged 20 and over have diabetes (2009-2012 National Health and Nutrition Examination Survey estimates applied to 2012 U.S. Census data). In adults 90 to 95% of the diabetes is type 2 diabetes.

There is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia. It has been shown that considerable weight loss can effectively cure type 2 diabetes (Lim et al, Diabetologia June 2011).

Although diet and exercise provide a simple method to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients. It is not only obese subjects who wish to lose weight. People with weight within the recommended range, for example in the upper part of the recommended range, may wish to reduce their weight, to bring it closer to their ideal weight. Thus, a need remains for agents that can be used to effect weight loss in overweight and obese subjects as well as subjects who are of normal weight.

A number of approaches to the development of agents useful in effecting weight loss have involved gastrointestinal peptide hormones and their analogues. Glucose-dependent insulinotropic polypeptide (GIP), also known as gastric inhibitory polypeptide or gastric inhibitory peptide, is a gastrointestinal hormone belonging to the class of molecules referred to as incretins. GIP is produced in vivo by K cells in the upper small intestine, in response to food. The main function of GIP is to induce the release of insulin from beta cells in the pancreas after eating. Increased insulin secretion leads to a decrease in circulating glucose concentration.

As well as its role as an incretin, GIP also inhibits apoptosis, and promotes proliferation, of the pancreatic beta cells. GIP is also known to stimulate glucagon secretion and fat accumulation. GIP receptors are found in other organs of the body, such as in the central nervous system, where GIP influences hippocampal memory formation and regulation of appetite and satiety. GIP is also proposed to play a part in bone remodelling, by increasing the formation of bone whilst decreasing bone breakdown.

Tirzepatide is a first-in-class medication that is a GIP-analogue which activates both the GLP-1 and GIP receptors and it was approved for medical use in the United States in May 2022 in the treatment of Type 2 diabetes.

However, despite significant advances, the process of identifying substances useful as drugs remains a complex and, in many cases, unpredictable field. In order to be useful as therapeutic agents, compounds must possess a suitable range of properties. In addition to having good efficacy at the biological target of interest, compounds must have good in vivo pharmacokinetic properties, low toxicity and an acceptable side effect profile. For example, even with commercial agents such as liraglutide, side effects can include nausea and vomiting, and concerns have also been raised with regard to thyroid cancer and pancreatitis.

Thus, there remains a need for further compounds which are useful for the treatment of disorders and diseases such as diabetes and obesity. For example, it would be desirable to identify peptides having beneficial properties such as an improved activity profile, and/or which have reduced side effects. For example, it would be desirable for a peptide to be identified that increases energy expenditure in a subject, but does not significantly reduce food intake. If a compound decreases food intake less, then it is expected that the compound will have fewer side effects such as nausea. Alternatively, or additionally, it would be desirable for a peptide to be identified that has these and other biological effects for a sustained period. A compound that has a longer period of activity can be administered less frequently and at lower dose, which contributes to improved convenience for the subject, to fewer side effects and to lower cost.

### SUMMARY OF THE INVENTION

In a first aspect of the invention there is provided a compound of formula:

[SEQ ID NO: 1]
Xaa1-Xaa2-Xaa3-Gly4-Thr5-Phe6-Xaa7-Ser8-Asp9-Xaa10-Ser11-Xaa12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-Xaa30-Xaa31-Xaa32-Xaa33-Xaa34-Xaa35-
Xaa36-Xaa37-Xaa38-Xaa39-Xaa40-Thr41-Gln42-Xaa43

wherein:
Xaa1 is DTyr, His, Phe or Tyr, or absent;
Xaa2 is AIB or Ala, or absent;
Xaa3 is Asp, Gln, Glu or His;
Xaa7 is IIe, Thr or Ser;
Xaa10 is Tyr or His;
Xaa12 is IIe or Lys;
Xaa13 is AIB, Ala, Gln, Glu, Gly, His, Pro, Ser, Thr, Tyr, Val, Asp or Asn;
Xaa14 is Leu, Met or NLeu;
Xaa17 is Gln or IIe;
Xaa18 is Ala, Arg or His;
Xaa19 is Ala or Gln;
Xaa20 is AIB, Gln, His, or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn;
Xaa29 is Gln, Gly or Thr;
Xaa30 is Gly, Lys or Lys*, or absent;
Xaa31 is Arg, Gly or Pro, or absent;
Xaa32 is Asn, Lys or Lys*, or absent;
Xaa33 is Lys or Lys*, or absent;
Xaa34 is Asn or Lys*;
Xaa35 is Asp or absent;
Xaa36 is Trp or Lys*, or absent;
Xaa37 is Lys or Lys*, or absent;
Xaa38 is His or absent;
Xaa39 is Asn or Lys*, or absent;
Xaa40 is IIe, Leu or Lys*, or absent;
Xaa43 is Lys* or absent;
   wherein Lys* is Lys substituted at its ε-amino group with a group of the formula:

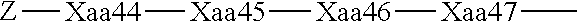

wherein:
Xaa44 is Gly, Ser, Thr or absent;
Xaa45 is Gly, Ser or absent;
Xaa46 is Asn, Gln, Gly, Ser, Thr or absent;
Xaa47 is Asn, Gln, Glu, Gly, His, Lys, Pro, Ser, Thr or absent;
and Z is a group of the formula:

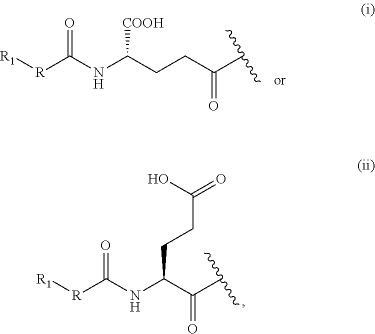

wherein R is a C₈-C₂₈ alkylene or alkenylene chain and R¹ is —CO₂H; and wherein the compound of formula (I) or formula (II) contains one Lys* residue; or a derivative of the compound; or a salt or solvate of the compound or the derivative.

Also provided herein is a composition comprising a compound, derivative, salt or solvate of the invention together with a pharmaceutically acceptable carrier and optionally a further therapeutic agent.

Also provided herein is a compound, derivative, salt or solvate of the invention, or a composition comprising such a compound, derivative, salt or solvate and a pharmaceutically acceptable carrier, for use as a medicament, e.g. for use in the prevention or treatment of diabetes, obesity, heart disease, stroke or non-alcoholic fatty liver disease, improving insulin release in a subject, improving carbohydrate metabolism in a subject, improving the lipid profile of a subject, reducing appetite, reducing food intake, reducing calorie intake, improving carbohydrate tolerance in a subject, and/or for use as a cytoprotective agent, for example in the prevention or treatment of Parkinsonism, Alzheimer's disease and other types of neural and cellular degeneration.

Also provided herein is a method of treating or preventing a disease or disorder or other non-desired physiological state in a subject comprising administration of a therapeutically effective amount of a compound, derivative, salt or solvate of the invention, or of a composition comprising such a compound, derivative, salt or solvate and a pharmaceutically acceptable carrier, e.g. in a method of treating or preventing diabetes, obesity, heart disease, stroke or non-alcoholic fatty liver disease in a subject, improving insulin release in a subject, improving carbohydrate metabolism in a subject, improving the lipid profile of a subject, improving carbohydrate tolerance in a subject, reducing appetite, reducing food intake, reducing calorie intake, and/or providing cytoprotection in a subject.

Also provided herein is a use of a compound, derivative, salt or solvate of the invention for the manufacture of a medicament for the prevention or treatment of diabetes, obesity, heart disease, stroke or non-alcoholic fatty liver disease, improving insulin release in a subject, improving carbohydrate metabolism in a subject, improving the lipid profile of a subject, improving carbohydrate tolerance in a subject, reducing appetite, reducing food intake, reducing calorie intake, and/or for use as a cytoprotective agent, for example in the prevention or treatment of Parkinsonism, Alzheimer's disease and other types of neural and cellular degeneration.

Also provided herein is a method of causing weight loss or preventing weight gain in a subject for cosmetic purposes comprising administration of an effective amount of a compound, derivative, salt or solvate of the invention.

### BRIEF DESCRIPTION OF THE DRAWINGS

FIG. 1 shows the amino acid sequences of example compounds of the invention. The compounds are presented with the N-terminal residue at the left-hand side of the table (signified by the column titled “1”).

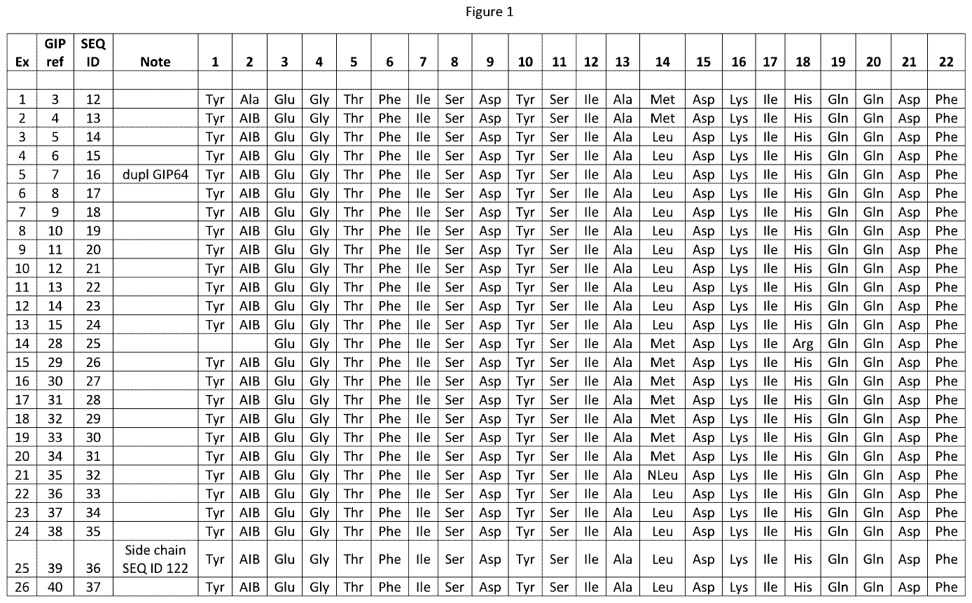

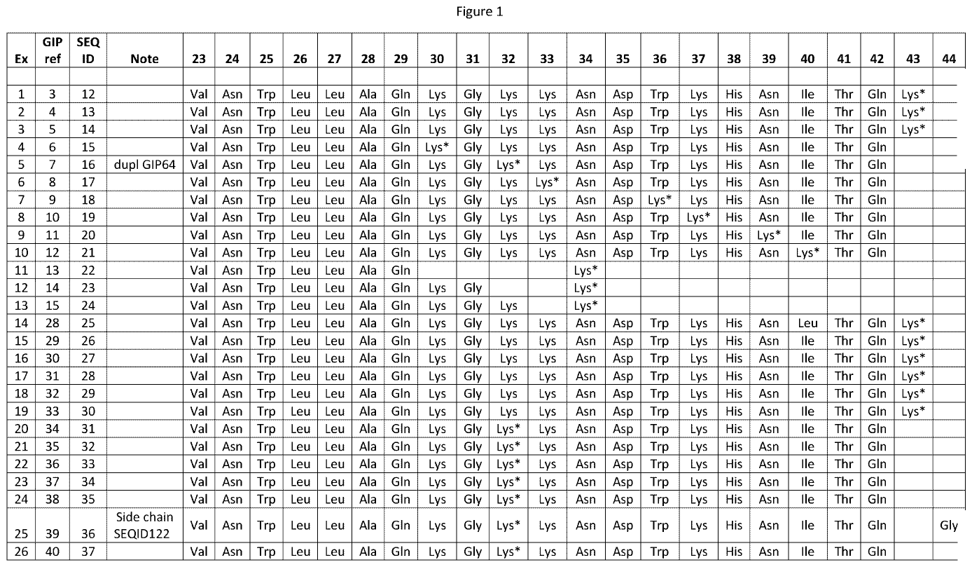

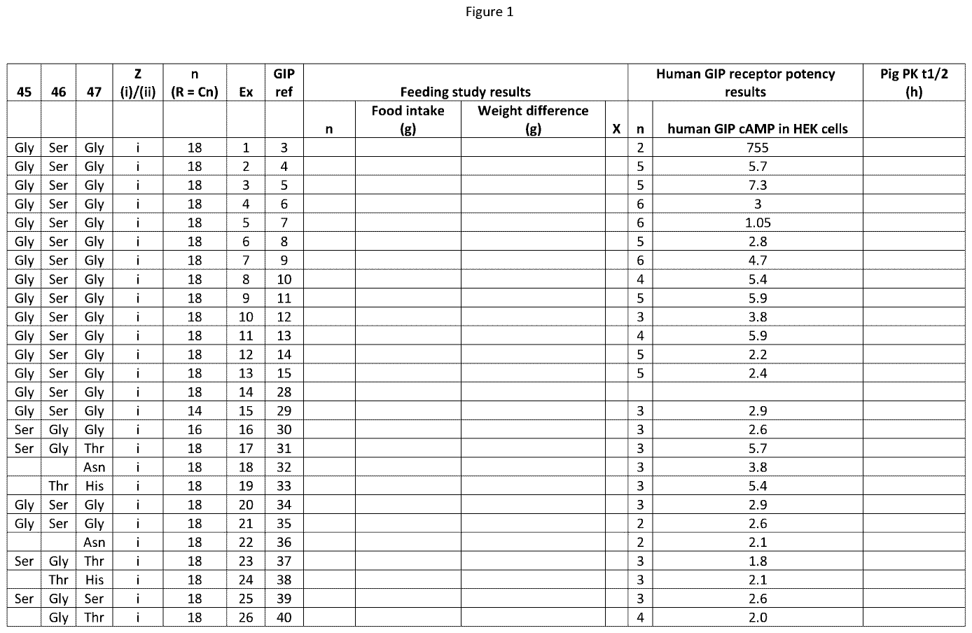

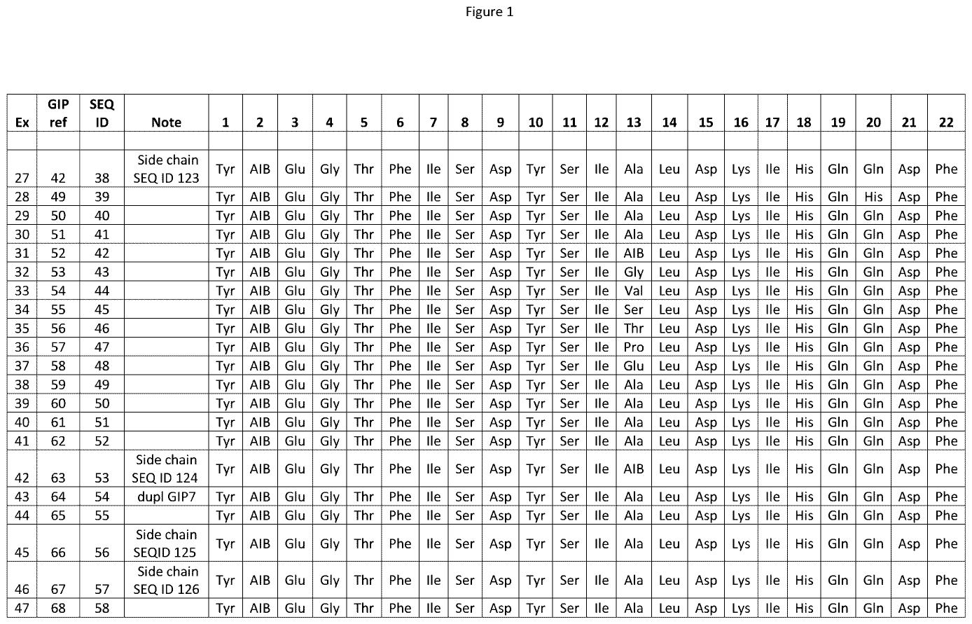

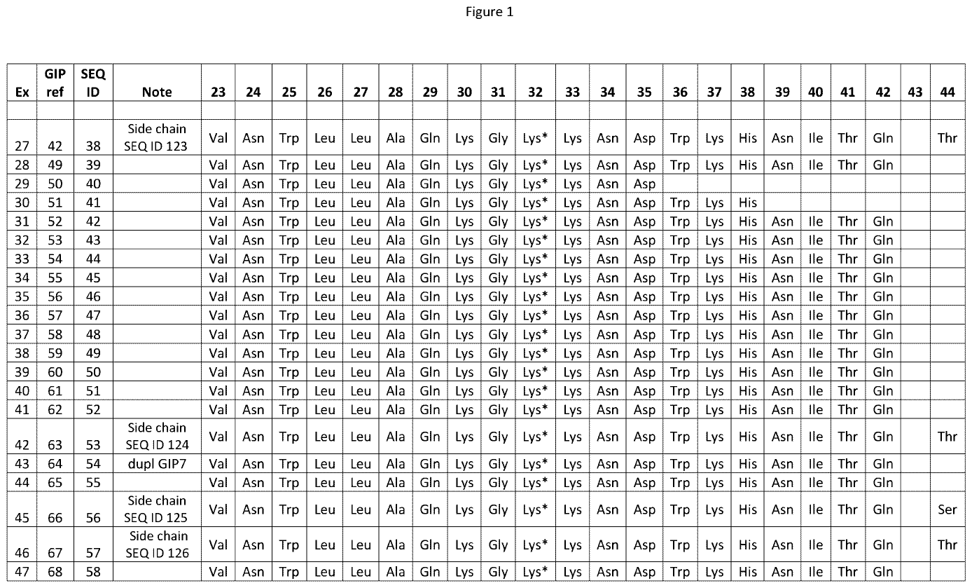

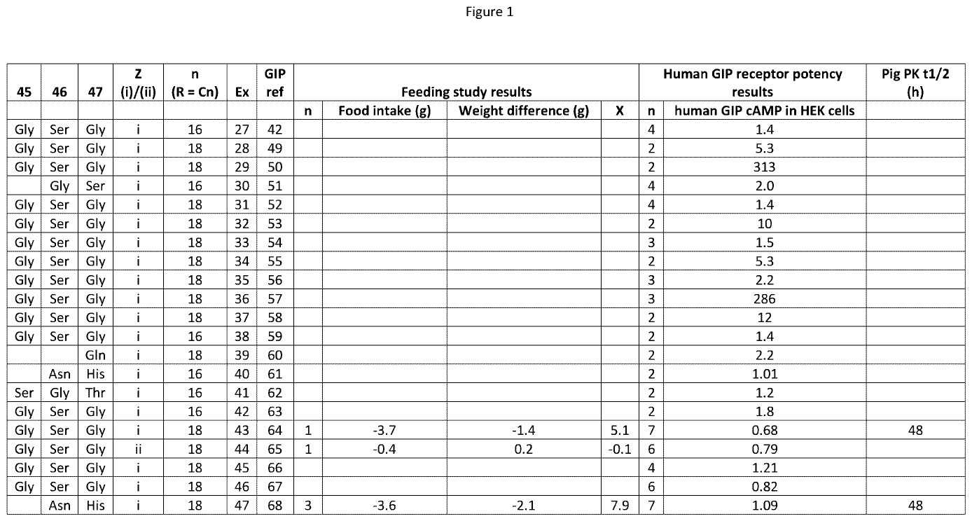

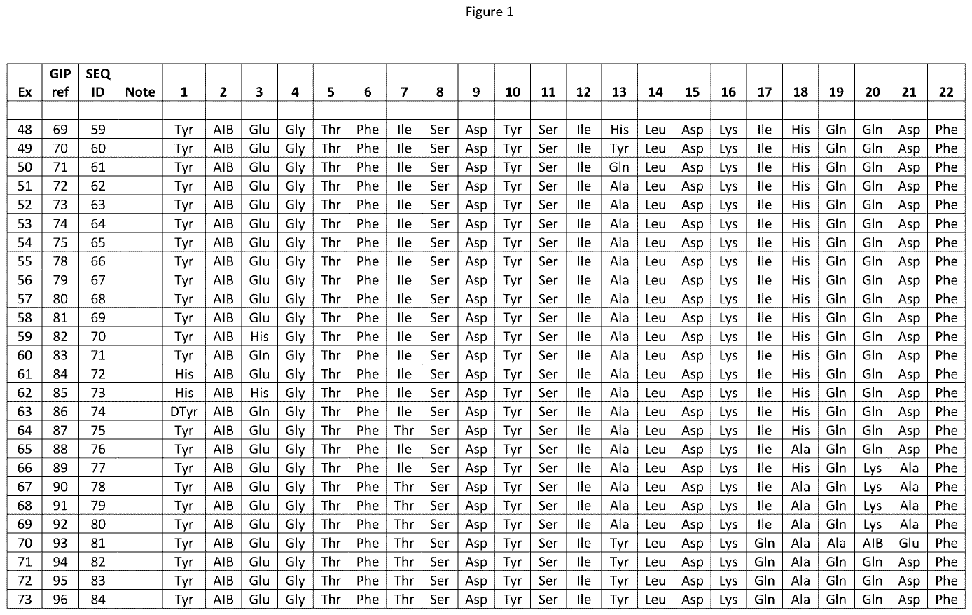

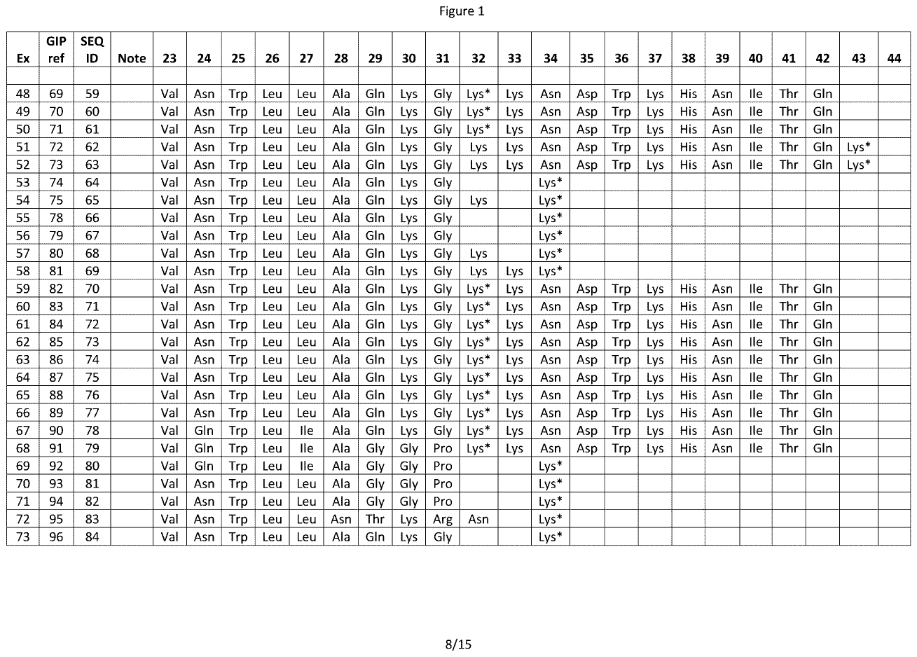

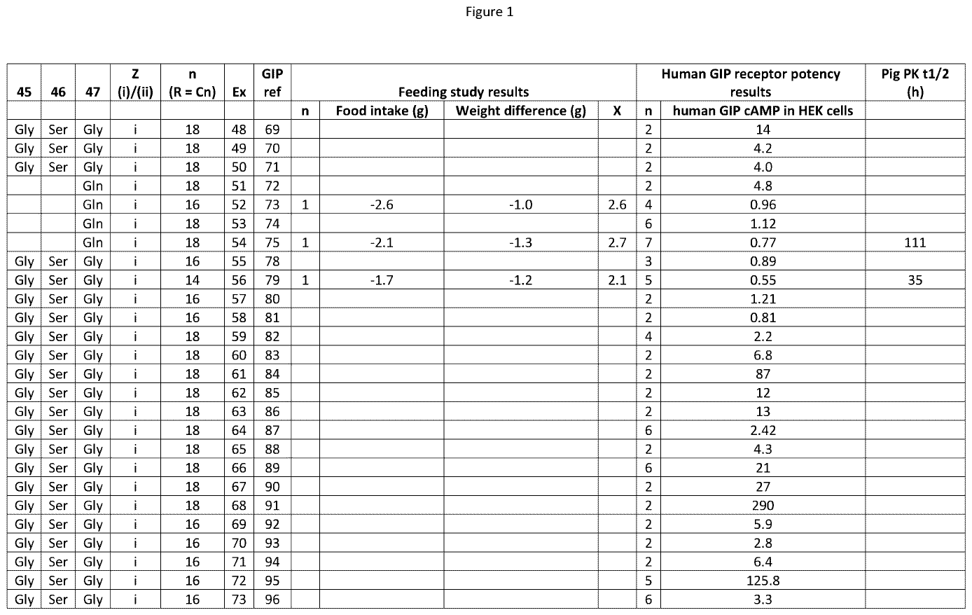

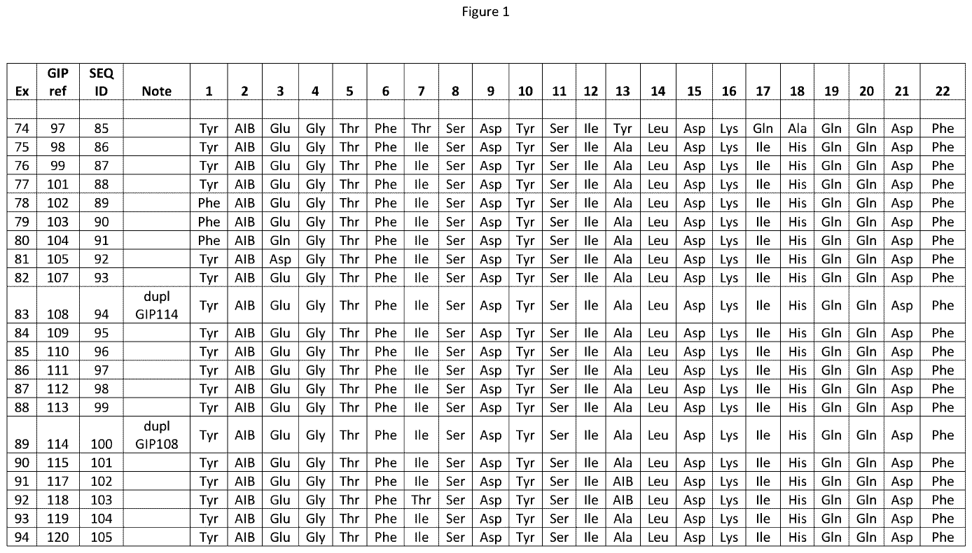

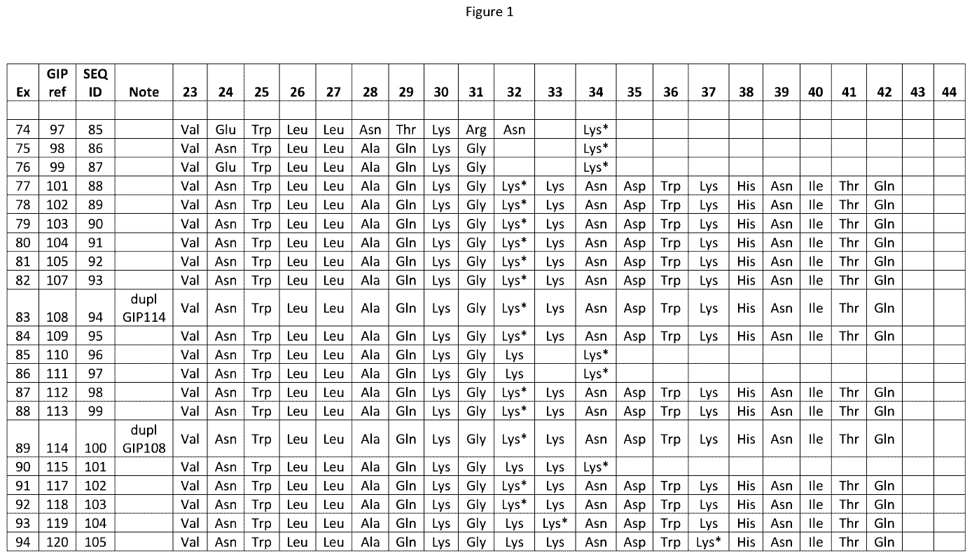

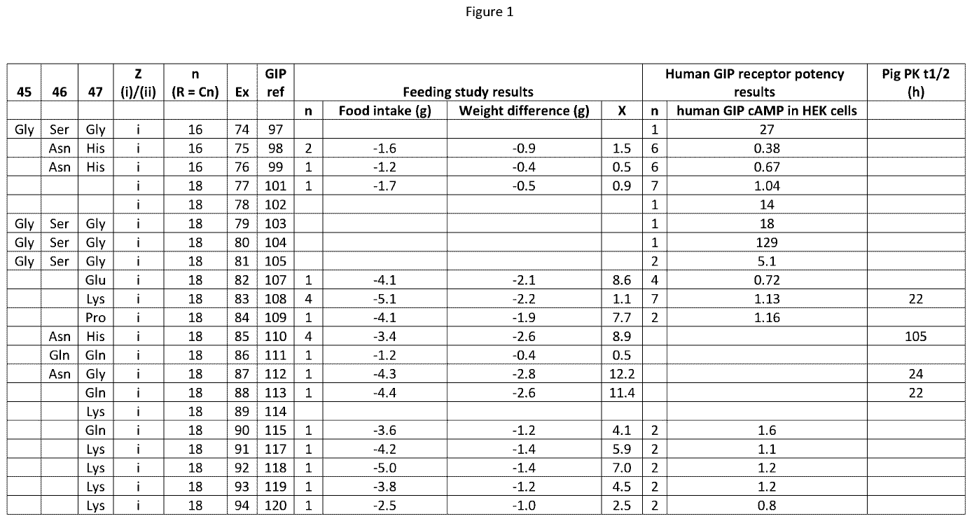

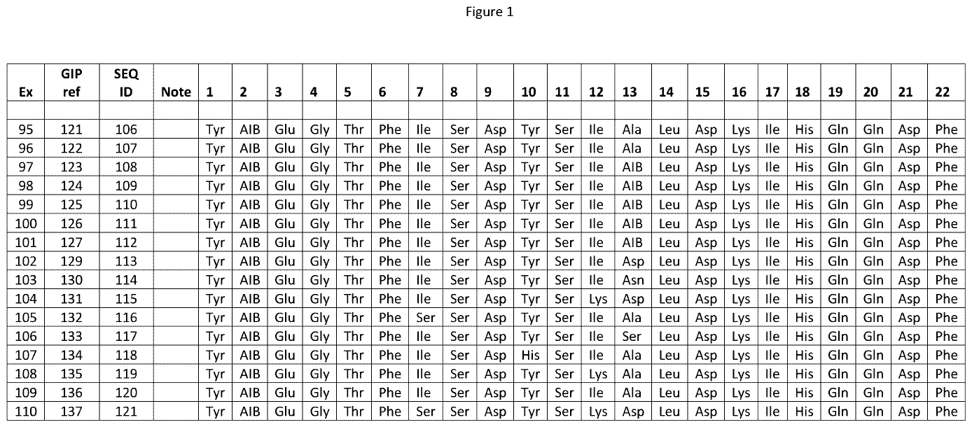

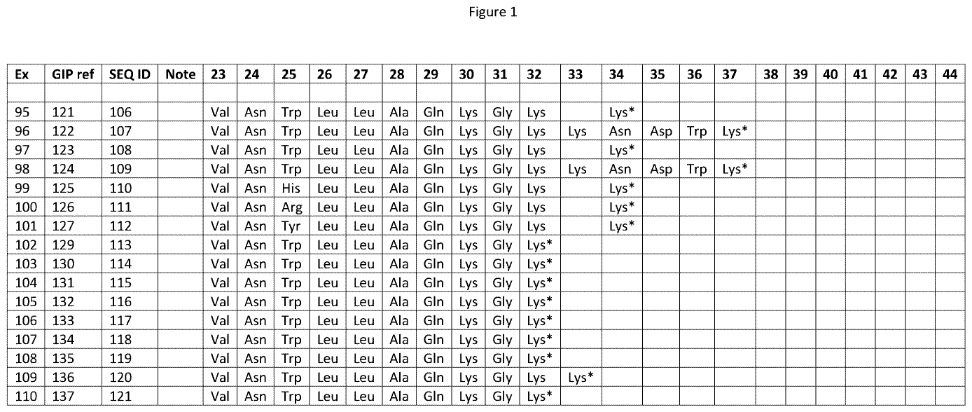

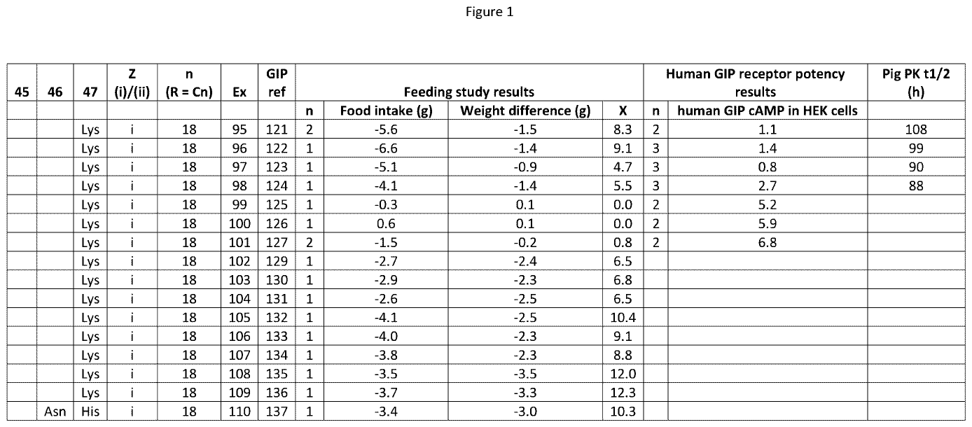

Results of receptor activation assay (reporter assay), feeding and pharmacokinetic experiments are shown in the far right hand columns of FIG. 1.

### Sequences

The amino acid sequences herein are shown with the N-terminus to the left, and where sequences are set out across multiple lines, the N-terminus is to the top left. Unless indicated otherwise, the amino acid residues in the sequences are L-amino acids.

The amino acid sequences listed in the application are shown using standard letter abbreviations for amino acids. The unnatural amino acid 2-aminoisobutyric acid has its usual abbreviation ‘AIB’. Norleucine is given the abbreviation ‘NLeu’.

The specific sequences given herein relate to specific embodiments of the invention.

### DETAILED DESCRIPTION

### Definitions

In order to facilitate review of the various embodiments of this disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and veterinary subjects. In preferred embodiments of the invention, the subject is a human subject.

Appetite: A natural desire, or longing for food. In one embodiment, appetite is measured by a survey to assess the desire for food. Increased appetite generally leads to increased feeding behaviour.

Appetite Suppressants: Compounds that decrease the desire for food. Commercially available appetite suppressants include, but are not limited to, amfepramone (diethylpropion), phentermine, mazindol and phenylpropanolamine fenfluramine, dexfenfluramine, and fluoxetine.

Body Mass Index (BMI): A mathematical formula for measuring body mass, also sometimes called Quetelet's Index. BMI is calculated by dividing weight (in kg) by height² (in metres²). The current standards for both men and women accepted as “normal” are a BMI of 20-24.9 kg/m². In one embodiment, a BMI of greater than 25 kg/m² can be used to identify an obese subject. Grade I obesity (which is sometimes referred to as being “overweight” rather than obesity) corresponds to a BMI of 25-29.9 kg/m². Grade II obesity corresponds to a BMI of 30-40 kg/m²; and Grade III obesity corresponds to a BMI greater than 40 kg/m² (Jequier, Am. J Clin. Nutr. 45:1035-47, 1987). Ideal body weight will vary among species and individuals based on height, body build, bone structure, and sex.

Cardioprotection refers to the protection of cardiac cells (and especially the myocardial cells) from apoptosis, necrotic cell death or degeneration (loss of function).

Cardioprotection is most often required following myocardial infarction, but may also be used in subjects suffering from ischemic heart disease (for example angina)

Cytoprotection refers to the protection of cells from apoptosis, necrotic cell death or degeneration (loss of function).

Diabetes: A failure of cells to transport endogenous glucose across their membranes either because of an endogenous deficiency of insulin and/or a defect in insulin sensitivity. Diabetes is a chronic syndrome of impaired carbohydrate, protein, and fat metabolism owing to insufficient secretion of insulin or to target tissue insulin resistance. It occurs in two major forms: insulin-dependent diabetes mellitus (IDDM, type I) and non-insulin dependent diabetes mellitus (NIDDM, type II) which differ in etiology, pathology, genetics, age of onset, and treatment.

The two major forms of diabetes are both characterized by an inability to deliver insulin in an amount and with the precise timing that is needed for control of glucose homeostasis. Diabetes type I, or insulin dependent diabetes mellitus (IDDM) is caused by the destruction of B cells, which results in insufficient levels of endogenous insulin. Diabetes type II, or non-insulin dependent diabetes, results from a defect in both the body's sensitivity to insulin, and a relative deficiency in insulin production.

Energy Metabolism: The body has to expend a certain amount of energy to maintain normal metabolism. In civilized man this is often set at about 2,800 Calories daily. If food consumption does not provide this, weight loss results. However, energy metabolism is also regulated, and, for example, administration of glucagon is thought to increase the metabolic rate so that a greater food intake is required to achieve energy balance and maintain weight. Thus, if food intake is maintained at the usual level, but energy metabolism is increased, weight loss will result.

Food intake: The amount of food consumed by an individual. Food intake can be measured by volume or by weight. For example, food intake may be the total amount of food consumed by an individual. In a feeding experiment, ‘Food Intake’ is the weight of a standardised chow consumed by an animal in a 24 hour period. Or, food intake may be the amount of proteins, fat, carbohydrates, cholesterol, vitamins, minerals, or any other food component, of the individual. “Protein intake” refers to the amount of protein consumed by an individual. Similarly, “fat intake,” “carbohydrate intake,” “cholesterol intake,” “vitamin intake,” and “mineral intake” refer to the amount of proteins, fat, carbohydrates, cholesterol, vitamins, or minerals consumed by an individual.

GIP: Glucose-dependent insulinotropic polypeptide (GIP), also known as gastric inhibitory polypeptide or gastric inhibitory peptide, is a 42-amino acid peptide released by the K cells of the duodenum and jejunum in response to food intake. GIP, together with GLP-1, are members of the hormone peptide class of incretins.

The sequence of human GIP is: Tyr-Ala-Glu-Gly-Thr-Phe-IIe-Ser-Asp-Tyr-Ser-IIe-Ala-Met-Asp-Lys-IIe-His-Gln-Gln-Asp-Phe-Val-Asn-Trp-Leu-Leu-Ala-Gln-Lys-Gly-Lys-Lys-Asn-Asp-Trp-Lys-His -Asn-IIe-Thr-Gln [SEQ ID NO: 2].

GLP-1: Glucagon-like peptide 1 (GLP-1) is derived from the transcription product of the proglucagon gene. The biologically active forms of GLP-1 are truncated forms known as GLP-1 (7-37) and GLP-1 (7-36)—NH₂ (the designation-NH₂ designates an amino acid sequence in which the C-terminal amino acid has a —C(O)NH₂ group in place of a carboxylic acid group).

The sequence of human GLP-1 (7-37) is His-Ala-Glu-Gly-Thr-Phe-Thr-Ser-Asp-Val-Ser-Ser-Tyr-Leu-Glu-Gly-Gln-Ala-Ala-Lys-Glu-Phe-IIe-Ala-Trp-Leu-Val-Lys-Gly-Arg-Gly [SEQ ID NO: 3].

The sequence of human GLP-1 (7-36)—NH₂ is His-Ala-Glu-Gly-Thr-Phe-Thr-Ser-Asp-Val-Ser-Ser-Tyr-Leu-Glu-Gly-Gln-Ala-Ala-Lys-Glu-Phe-IIe-Ala-Trp-Leu-Val-Lys-Gly-Arg-CONH₂ [SEQ ID NO: 4].

Glucagon: Glucagon is a peptide derived from the proglucagon gene. It is a 29-amino acid polypeptide in humans and has the sequence:

[SEQ ID NO: 5]
His-Ser-Gln-Gly-Thr-Phe-Thr-Ser-Asp-Tyr-Ser-Lys-Tyr-Leu-Asp-Ser-Arg-Arg-Ala-Gln-
Asp-Phe-Val-Gln-Trp-Leu-Met-Asn-Thr.

Neuroprotection refers to the protection of neurons within the nervous system (preferably within the central nervous system) from apoptosis, necrotic cell death or degeneration (loss of function). Neuroprotective treatments, including those relating to various aspects of the present invention may be required following a brain injury (for example those following physical trauma or non-traumatic injury such as stroke, brain tumours, infection, poisoning, hypoxia, ischemia, encephalopathy or substance abuse). Neuroprotective treatments, including those relating to various aspects of the present invention may also be indicated in subjects having a chronic neurodegenerative disease such as Alzheimer's disease, Parkinson's disease, Gehrig's disease or Huntington's disease.

Normal Daily Diet: The average food intake for an individual of a given species. A normal daily diet can be expressed in terms of caloric intake, protein intake, carbohydrate intake, and/or fat intake. A normal daily diet in humans generally comprises the following: about 2,000, about 2,400, or about 2,800 to significantly more calories. In addition, a normal daily diet in humans generally includes about 12 g to about 45 g of protein, about 120 g to about 610 g of carbohydrate, and about 11 g to about 90 g of fat. A low calorie diet would be no more than about 85%, and preferably no more than about 70%, of the normal caloric intake of a human individual.

In animals, the caloric and nutrient requirements vary depending on the species and size of the animal. For example, in cats, the total caloric intake per pound, as well as the percent distribution of protein, carbohydrate and fat varies with the age of the cat and the reproductive state. A general guideline for cats, however, is 40 cal/lb/day (18.2 cal/kg/day). About 30% to about 40% should be protein, about 7% to about 10% should be from carbohydrate, and about 50% to about 62.5% should be derived from fat intake. One of skill in the art can readily identify the normal daily diet of an individual of any species.

Obesity: A condition in which excess body fat may put a person at health risk (see Barlow and Dietz, Pediatrics 102: E29, 1998; National Institutes of Health, National Heart, Lung, and Blood Institute (NHLBI), Obes. Res. 6 (suppl. 2): 51S-209S, 1998). Excess body fat is a result of an imbalance of energy intake and energy expenditure. For example, the Body Mass Index (BMI) may be used to assess obesity. In one commonly used convention, a BMI of 25.0 kg/m² to 29.9 kg/m² is overweight, while a BMI of 30 kg/m² or greater is obese.

In another convention, waist circumference is used to assess obesity. In this convention, in men a waist circumference of 102 cm or more is considered obese, while in women a waist circumference of 89 cm or more is considered obese. Strong evidence shows that obesity affects both the morbidity and mortality of individuals. For example, an obese individual is at increased risk for heart disease, non-insulin dependent (type 2) diabetes, hypertension, stroke, cancer (e.g. endometrial, breast, prostate, and colon cancer), dyslipidemia, gall bladder disease, sleep apnea, reduced fertility, and osteoarthritis, amongst others (see Lyznicki et al., Am. Fam. Phys. 63:2185, 2001).

Overweight: An individual who weighs more than their ideal body weight. An overweight individual can be obese but is not necessarily obese. For example, an overweight individual is any individual who desires to decrease their weight. In one convention, an overweight individual is an individual with a BMI of 25.0 kg/m² to 29.9 kg/m².

Oxyntomodulin (OXM): Oxyntomodulin is a 37 amino acid peptide member of the glucagon superfamily comprising the entire 29 amino acid sequence of glucagon, with an eight amino acid carboxy terminal extension, resulting from the tissue-specific processing of the pre-pro-glucagon precursor in the brain and gut. The human OXM sequence is as follows:

[SEQ ID NO: 6]
His-Ser-Gln-Gly-Thr-Phe-Thr-Ser-Asp-Tyr-Ser-Lys-Tyr-Leu-Asp-Ser-Arg-Arg-Ala-Gln-
Asp-Phe-Val-Gln-Trp-Leu-Met-Asn-Thr-Lys-Arg-Asn-Arg-Asn-Asn-Ile-Ala.

PEGylation and PEGylated: PEGylation refers to the process of reacting a poly (alkylene glycol), preferably an activated poly (alkylene glycol) to form a covalent bond. A facilitator may be used, for example an amino acid, e.g. lysine. Although “PEGylation” is often carried out using poly(ethylene glycol) or derivatives thereof, such as methoxy poly(ethylene glycol), the term is not limited herein to the use of methoxy poly (ethylene glycol) but also includes the use of any other useful poly (alkylene glycol), for example poly(propylene glycol). The term PEGylated refers to a compound containing such a poly (alkylene glycol) group.

pl: pl is an abbreviation for isoelectric point. An alternative abbreviation sometimes used is IEP. It is the pH at which a particular molecule carries no net electric charge. At a pH below its pl a protein or peptide carries a net positive charge. At a pH above its pl a protein or peptide carries a net negative charge. Proteins and peptides can be separated according to their isoelectric points using a technique called isoelectric focusing which is an electrophoretic method that utilises a pH gradient contained within a polyacrylamide gel.

Peripheral administration: Administration outside of the central nervous system. Peripheral administration does not include direct administration to the brain. Peripheral administration includes, but is not limited to intravascular, intramuscular, subcutaneous, inhalation, oral, rectal, transdermal or intra-nasal administration.

Polypeptide: A polymer in which the monomers are amino acid residues which are joined together through amide bonds. Unless dictated otherwise by context, the terms “polypeptide”, “peptide”, or “protein” as used herein encompass any amino acid sequence and include modified sequences such as glycoproteins. The term “polypeptide” covers naturally occurring proteins, as well as those which are recombinantly or synthetically produced. The term “polypeptide fragment” refers to a portion of a polypeptide, for example a fragment which exhibits at least one useful sequence in binding a receptor. The term “functional fragments of a polypeptide” refers to all fragments of a polypeptide that retain an activity of the polypeptide. Biologically functional peptides can also include fusion proteins, in which the peptide of interest has been fused to another peptide that does not decrease its desired activity.

Subcutaneous administration: Subcutaneous administration is administration of a substance to the subcutaneous layer of fat which is found between the dermis of the skin and the underlying tissue. Subcutaneous administration may be by an injection using a hypodermic needle fitted, for example, to a syringe or a “pen” type injection device. Other administration methods may be used for example microneedles. Injection with a hypodermic needle typically involves a degree of pain on behalf of the recipient. Such pain may be masked by use of a local anaesthetic or analgesic. However, the usual method used to reduce the perceived pain of injections is to merely distract the subject immediately prior to and during the injection. Pain may be minimised by using a relatively small gauge hypodermic needle, by injecting a relatively small volume of substance and by avoiding excessively acidic or alkali compositions which may cause the subject to experience a “stinging” sensation at the injection site. Compositions having a pH of between pH 4 and pH 10 are usually regarded as tolerably comfortable.

Therapeutically effective amount: A dose sufficient to prevent advancement, or to cause regression of a disorder, or which is capable of relieving a sign or symptom of a disorder, or which is capable of achieving a desired result. In some embodiments, a therapeutically effective amount of a compound of the invention is an amount sufficient to inhibit or halt weight gain, or an amount sufficient to decrease appetite.

### Compounds of the Invention

The present inventors have found that example compounds of the invention have properties including causing weight loss in vivo. The compounds also have a long half-life in the blood, meaning that they can be administered at a conveniently low frequency.

Compared with GIP and previous derivatives of GIP, the compounds of the invention have a substituent at a lysine residue at one of a number of positions in the peptide sequence, the substituted lysine is functionalised at its ε-amino group, and the substituent group comprises a peptide sequence of up to 4 residues, terminating in the group R—R₁ where R¹ is CO₂H.

These are functionalisations that have not previously been investigated and the beneficial properties found by the current inventors have not previously been seen.

As described above, the compounds of the invention have formula (I):

[SEQ ID NO:1]
Xaa1-Xaa2-Xaa3-Gly4-Thr5-Phe6-Xaa7-Ser8-Asp9-Xaa10-Ser11-Xaa12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-Xaa30-Xaa31-Xaa32-Xaa33-Xaa34-Xaa35-
Xaa36-Xaa37-Xaa38-Xaa39-Xaa40-Thr41-Gln42-Xaa43

wherein:
   Xaa1 is DTyr, His, Phe or Tyr, or absent;
   Xaa2 is AIB or Ala, or absent;
   Xaa3 is Asp, Gln, Glu or His;
   Xaa7 is IIe, Thr or Ser;
   Xaa10 is Tyr or His;
   Xaa12 is IIe or Lys;
   Xaa13 is AIB, Ala, Gln, Glu, Gly, His, Pro, Ser, Thr, Tyr, Val, Asp or Asn;
   Xaa14 is Leu, Met or NLeu;
   Xaa17 is Gln or IIe;
   Xaa18 is Ala, Arg or His;
   Xaa19 is Ala or Gln;
   Xaa20 is AIB, Gln, His, or Lys;
   Xaa21 is Ala, Asp or Glu;
   Xaa24 is Asn, Gln or Glu;
   Xaa25 is Arg, His, Trp or Tyr;
   Xaa27 is IIe or Leu;
   Xaa28 is Ala or Asn;
   Xaa29 is Gln, Gly or Thr;
   Xaa30 is Gly, Lys or Lys*, or absent;
   Xaa31 is Arg, Gly or Pro, or absent;
   Xaa32 is Asn, Lys or Lys*, or absent;
   Xaa33 is Lys or Lys*, or absent;
   Xaa34 is Asn or Lys*;
   Xaa35 is Asp or absent;
   Xaa36 is Trp or Lys*, or absent;
   Xaa37 is Lys or Lys*, or absent;
   Xaa38 is His or absent;
   Xaa39 is Asn or Lys*, or absent;
   Xaa40 is IIe, Leu or Lys*, or absent;
   Xaa43 is Lys* or absent;
wherein Lys* is Lys substituted at its ε-amino group with a group of the formula:

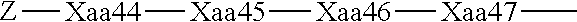

wherein:
Xaa44 is Gly, Ser, Thr or absent;
Xaa45 is Gly, Ser or absent;
Xaa46 is Asn, Gln, Gly, Ser, Thr or absent;
Xaa47 is Asn, Gln, Glu, Gly, His, Lys, Pro, Ser, Thr or absent;
and Z is a group of the formula:

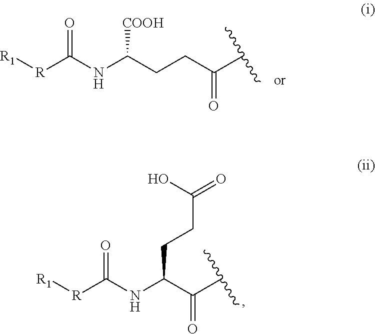

wherein R is a C₈-C₂₈ alkylene or alkenylene chain and R¹ is —CO₂H; and wherein the compound of formula (I) or formula (II) contains one Lys* residue.

Considering the residues in turn,
Xaa1 is selected from DTyr, His, Phe or Tyr, or Xaa1 is absent. Preferably, Xaa1 is selected from His, Phe and Tyr. For example, Xaa1 is selected from His and Tyr, or alternatively Xaa1 is selected from Phe and Tyr. For example, Xaa1 is Tyr. In an especially preferred embodiment, Xaa1 is Tyr.

Xaa2 is selected from AIB or Ala, or absent. Preferably, Xaa2 is AIB.

Xaa3 is selected from Asp, Gln, Glu or His. Preferably, Xaa3 is selected from Glu and Gln. For example, Xaa3 is Glu. Alternatively, Xaa3 is Gln. In an especially preferred embodiment, Xaa3 is Glu.

Xaa7 is selected from IIe, Thr or Ser. In one embodiment, Xaa7 is selected from IIe or Thr. In another embodiment, Xaa7 is selected from IIe or Ser. Preferably, Xaa7 is IIe. Xaa10 is selected from Tyr or His. Preferably, Xaa 10 is Tyr.

Xaa12 is selected from IIe or Lys. Preferably, Xaa12 is IIe.

Xaa13 is selected from AIB, Ala, Gln, Glu, Gly, His, Pro, Ser, Thr, Tyr, Val, Asp or Asn. In an embodiment, Xaa13 is selected from AIB, Ala, Gln, Glu, Gly, His, Pro, Ser, Thr, Tyr or Val. Preferably, Xaa13 is selected from Ala, Gln, Thr and Val. For example, Xaa3 is selected from Ala, Gln, and Val. For example, Xaa13 is selected from Ala and Gln, or alternatively Xaa13 is selected from Ala and Val. In an especially preferred embodiment, Xaa13 is Ala.

In another embodiment, Xaa13 is selected from Ala, Asp, Asn, Ser. In an especially preferred embodiment, Xaa13 is Ala.

According to certain embodiments, Xaa7 is selected from IIe or Thr, Xaa10 is Tyr, Xaa12 is IIe and Xaa13 is selected from AIB, Ala, Gln, Glu, Gly, His, Pro, Ser, Thr, Tyr or Val.

Xaa14 is selected from Leu, Met or NLeu. Preferably, Xaa14 is Leu.

Xaa17 is selected from Gln or IIe. Preferably, Xaa17 is IIe.

Xaa18 is selected from Ala, Arg or His. Preferably, Xaa18 is selected from Ala and His. For example, Xaa18 is His. Alternatively, Xaa18 is Ala. In an especially preferred embodiment, Xaa18 is His.

Xaa19 is selected from Ala or Gln. Preferably, Xaa19 is Gln.

Xaa20 is selected from AIB, Gln, His or Lys. Preferably, Xaa20 is selected from AIB, Gln, and His. For example, Xaa20 is selected from AIB and Gln, or alternatively Xaa20 is selected from Gln and His. In an especially preferred embodiment, Xaa20 is Gln. Xaa21 is selected from Ala, Asp or Glu. Preferably, Xaa21 is selected from Asp and Glu. For example, Xaa21 is Asp. Alternatively, Xaa21 is Glu. In an especially preferred embodiment, Xaa21 is Asp.

Xaa24 is selected from Asn, Gln or Glu. Preferably, Xaa24 is selected from Asn and Gln. For example, Xaa24 is Asn. Alternatively, Xaa24 is Gln. In an especially preferred embodiment, Xaa24 is Asn.

Xaa25 is selected from Arg, His, Trp or Tyr. Preferably, Xaa25 is Trp. Xaa27 is selected from IIe or Leu. Preferably, Xaa27 is Leu.

Xaa28 is selected from Ala or Asn. Preferably, Xaa28 is Ala.

Xaa29 is selected from Gln, Gly or Thr. Preferably, Xaa29 is selected from Gln and Gly. For example, Xaa29 is Gln. Alternatively, Xaa29 is Gly. In an especially preferred embodiment, Xaa29 is Gln.

Xaa30 is selected from Gly, Lys or Lys*, or Xaa30 is absent. Preferably, Xaa30 is selected from Lys and Gly. For example, Xaa30 is Lys. Alternatively, Xaa30 is Gly. In an especially preferred embodiment, Xaa30 is Lys.

Xaa31 is selected from Arg, Gly or Pro, or Xaa31 is absent. Preferably, Xaa31 is selected from Gly and Pro. For example, Xaa31 is Gly. Alternatively, Xaa31 is Pro. In an especially preferred embodiment, Xaa31 is Gly.

Xaa32 is selected from Asn, Lys or Lys*, or Xaa32 is absent. Preferably, Xaa32 is selected from Asn, Lys and Lys*. For example, Xaa32 is selected from Lys and Lys*. Alternatively, Xaa32 is selected from Asn and Lys*. In an especially preferred embodiment, Xaa32 is Lys*.

Xaa33 is Lys or Lys*; or Xaa33 is absent. Preferably, Xaa33 is selected from Lys and Lys*. Alternatively, Xaa33 is Lys or absent. In an especially preferred embodiment, Xaa33 is Lys.

Xaa34 is Asn or Lys*; or Xaa34 is absent. Preferably, Xaa34 is selected from Asn and Lys*, or Xaa34 is absent. For example, Xaa34 is selected from Asn and Lys*. Alternatively, Xaa34 is Asn or absent. In an especially preferred embodiment, Xaa34 is Asn.

Xaa35 is Asp; or Xaa35 is absent. For example, Xaa35 is Asp. Alternatively, Xaa35 is absent. In a preferred embodiment, Xaa35 is Asp.

Xaa36 is Trp or Lys*; or Xaa36 is absent. Preferably, Xaa36 is Trp or absent. For example, Xaa36 is Trp. Alternatively, Xaa36 is absent. In an especially preferred embodiment, Xaa35 is Trp.

Xaa37 is Lys or Lys*; or Xaa37 is absent. Preferably, Xaa37 is Lys or absent. For example, Xaa37 is Lys. Alternatively, Xaa37 is absent. In an especially preferred embodiment, Xaa37 is Lys.

Xaa38 is His or Xaa38 is absent. For example, Xaa38 is His. Alternatively, Xaa38 is absent. In an especially preferred embodiment, Xaa38 is His.

Xaa39 is Asn or Lys*; or Xaa39 is absent. Preferably, Xaa39 is Asn or absent. For example, Xaa39 is Asn. Alternatively, Xaa39 is absent. In an especially preferred embodiment, Xaa39 is Asn.

Xaa40 is IIe, Leu or Lys*; or Xaa40 is absent. Preferably, Xaa40 is IIe or absent. For example, Xaa40 is IIe. Alternatively, Xaa40 is absent. In an especially preferred embodiment, Xaa40 is IIe.

Xaa41 is Thr, or Xaa41 is absent. Preferably, Xaa41 is Thr.

Xaa42 is Gln, or Xaa42 is absent. Preferably, Xaa42 is Gln.

Xaa43 is Lys*, or Xaa43 is absent. Preferably, Xaa43 is absent.

### Attachment of Lys* Substituent

In the compounds of the invention, the ε-amino group on Lys* is attached to the α-acid group of the Xaa47 residue of the molecule, such that the bond is an amide bond. If Xaa47 is absent, then the ε-amino group on Lys* is attached to the α-acid group of the next residue that is present, or the α-acid group of the Z group if no residue is present.

In a lysine residue, the ε-amino group is the amino group that is attached to the 6-carbon. Following standard IUPAC nomenclature, the atoms in lysine are numbered as follows, indicating the carbon atom numbering and also the α to ε positions:

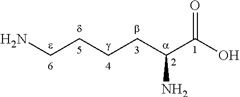

The ε-amino group on Lys* that is referred to herein is the amino group on the C-6 carbon atom as indicated.

According to the invention, the substituent may be attached to a Lys residue at any of the positions in the peptide sequence that is indicated in formula (I) by Lys*. That is to say, the substituent may be attached to a Lys residue at one of positions Xaa30, Xaa32, Xaa33, Xaa34, Xaa36, Xaa37, Xaa39, Xaa40 or Xaa43. Preferred positions are selected from Xaa30, Xaa32, Xaa33, Xaa34 and Xaa43, for example Xaa32, Xaa33 and Xaa43. In an especially preferred embodiment, the substituent is attached to a Lys residue at position Xaa32.

The substituent group in compounds of the invention is of the formula:

Z-Xaa44-Xaa45-Xaa46-Xaa47—

Considering each residue in turn:

Xaa44 is selected from Gly, Ser and Thr; or Xaa44 is absent. Preferably, Xaa44 is Thr, or Xaa44 is absent. For example Xaa44 is Thr, or alternatively Xaa44 is absent. In an especially preferred embodiment, Xaa44 is absent.

Xaa45 is selected from Gly and Ser; or Xaa45 is absent. For example, Xaa45 is Gly or absent. Alternatively, Xaa45 is Ser or absent. Preferably Xaa45 is Gly or absent. In an especially preferred embodiment, Xaa45 is absent.

Xaa46 is selected from Asn, Gln, Gly, Ser and Thr; or Xaa46 is absent. Preferably, Xaa46 is selected from Asn, Gly, Ser and Thr; or Xaa46 is absent. More preferably, Xaa46 is selected from Asn, Gly and Ser; or Xaa46 is absent. For example, Xaa46 is selected from Asn and Ser; or Xaa46 is absent. Alternatively, Xaa46 is selected from Gly and Ser; or Xaa46 is absent. In a more preferred embodiment, Xaa46 is Ser or absent. In an especially preferred embodiment, Xaa46 is absent.

Xaa47 is selected from Asn, Gln, Glu, Gly, His, Lys, Pro, Ser and Thr; or Xaa47 is absent. Preferably, Xaa47 is selected from Gly, Gln, His and Lys; or Xaa47 is absent. More preferably, Xaa47 is selected from Gly and Lys; or Xaa47 is absent. In a preferred embodiment, Xaa47 is Lys or absent. In an especially preferred embodiment, Xaa47 is Lys.

### Group Z

The Z portion of the compounds is a group of the formula:

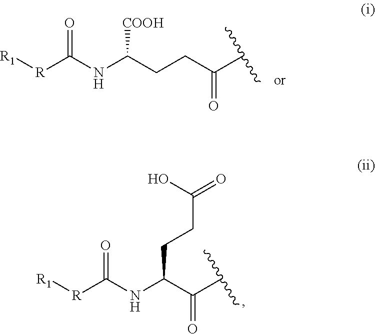

wherein R is a C₈-C₂₈ alkylene or alkenylene chain and R¹ is —CO₂H.

Within the Z portion of the molecule, group R is an alkylene or alkenylene chain, which is linked at one end to residue Xaa44 (or, if Xaa44 is absent, Xaa45, or if both Xaa44 and Xaa45 are absent, Xaa46, and so on; if all of Xaa44 to Xaa47 are absent, R is linked at one end to Lys*) by the Glu residue through an amide bond. At its other end, the R alkylene or alkenylene chain is linked to the R₁ acid group (CO₂H). Option (i) above represents the situation wherein the Glu residue in the Z group is attached to the Lys* residue through the γ-carboxylic acid group of Glu. Option (ii) above represents the situation wherein the Glu residue in the Z group is attached to the Lys* residue through the α-carboxylic acid group of Glu. In a preferred embodiment of the invention, the Z portion of the compound is of formula (i), i.e. the Glu residue in the Z group is attached to the Lys* residue through the γ-carboxylic acid group of Glu.

Generally, R has an even number of carbon atoms. For example, R can be an alkylene or alkenylene chain that is found in naturally-occurring fatty acids. The root fatty acid has a chain length two higher than the number of carbon atoms in the R alkylene or alkenylene chain.

In a preferred embodiment, R is a C₁₆-C₁₈ alkylene or alkenylene group. For example, R is straight chain alkylene or alkenylene group. For example, R is a C₁₆ or C₁₈ straight chain alkylene group. For example, when R is a C₁₆ group, it can be provided by an octadecanedioic acid moiety. For example, when R is a C₁₈ group, it can be provided by an eicosanedioic acid moiety.

In a more preferred embodiment, R is a C₁₈ alkylene group.

In an embodiment of the invention, it is preferred that:
Xaa1 is DTyr, His, Phe or Tyr, or absent;
Xaa2 is AIB or Ala, or absent;
Xaa3 is Asp, Gln, Glu or His;
Xaa7 is IIe or Thr;
Xaa10 is Tyr;
Xaa12 is IIe;
Xaa13 is AIB, Ala, Gln, Glu, Gly, His, Pro, Ser, Thr, Tyr or Val;
Xaa14 is Leu, Met or NLeu;
Xaa17 is Gln or IIe;
Xaa18 is Ala, Arg or His;
Xaa19 is Ala or Gln;
Xaa20 is AIB, Gln, His, or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn;
Xaa29 is Gln, Gly or Thr;
Xaa30 is Gly, Lys or Lys*, or absent;
Xaa31 is Arg, Gly or Pro, or absent;
Xaa32 is Asn, Lys or Lys*, or absent;
Xaa33 is Lys or Lys*, or absent;
Xaa34 is Asn or Lys*;
Xaa35 is Asp or absent;
Xaa36 is Trp or Lys*, or absent;
Xaa37 is Lys or Lys*, or absent;
Xaa38 is His or absent;
Xaa39 is Asn or Lys*, or absent;
Xaa40 is IIe, Leu or Lys*, or absent;
Xaa43 is Lys* or absent;
   wherein Lys* is Lys substituted at its ε-amino group with a group of the formula:

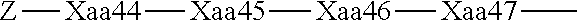

wherein:
Xaa44 is Gly, Ser, Thr or absent;
Xaa45 is Gly, Ser or absent;
Xaa46 is Asn, Gln, Gly, Ser, Thr or absent;
Xaa47 is Asn, Gln, Glu, Gly, His, Lys, Pro, Ser, Thr or absent;
and Z is a group of the formula:

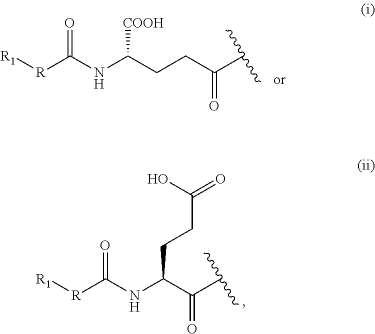

wherein R is a C₈-C₂₈ alkylene or alkenylene chain and R¹ is —CO₂H;
and wherein the compound of formula (I) or formula (II) contains one Lys* residue.

In an embodiment of the invention, it is preferred that:
Xaa1 is His, Phe or Tyr;
Xaa2 is AIB;
Xaa3 is Glu, Gln or His;
Xaa7 is IIe;
Xaa10 is Tyr;
Xaa12 is IIe;
Xaa13 is Ala, Gln, Thr or Val;
Xaa14 is Leu or Met;
Xaa17 is Gln or IIe;
Xaa18 is Ala or His;
Xaa19 is Ala or Gln;
Xaa20 is AIB, Gln, His or Lys;
Xaa21 is Asp or Glu;
Xaa24 is Asn, Gln or Glu;
Xaa25 is Trp;
Xaa27 is Leu;
Xaa28 is Ala or Asn;
Xaa29 is Gln, Gly or Thr;
Xaa30 is Gly, Lys or Lys*;
Xaa31 is Arg, Gly or Pro;
Xaa32 is Asn, Lys or Lys*;
Xaa33 is Lys or Lys*; or Xaa33 is absent;
Xaa34 is Asn or Lys*; or Xaa34 is absent;
Xaa35 is Asp; or Xaa35 is absent;
Xaa36 is Trp; or Xaa36 is absent;
Xaa37 is Lys; or Xaa37 is absent;
Xaa38 is His; or Xaa38 is absent;
Xaa39 is Asn; or Xaa39 is absent;
Xaa40 is IIe; or Xaa40 is absent;
Xaa41 is Thr; or Xaa41 is absent;
Xaa42 is Gln, or Xaa42 is absent;
Xaa43 is Lys*; or Xaa43 is absent;
Z is (i):

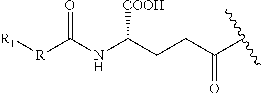

wherein R is a C₁₆ or C₁₈ alkylene chain and R¹ is —CO₂H;
Xaa44 is Thr, or Xaa44 is absent;
Xaa45 is Gly, or Xaa45 is absent;
Xaa46 is Asn, Gly or Ser, or Xaa46 is absent; and
Xaa47 is Gly or Lys, or Xaa47 is absent.

In a more preferred embodiment:
Xaa1 is Tyr;
Xaa2 is AIB;
Xaa3 is Glu or Gln;
Xaa7 is IIe;
Xaa10 is Tyr;
Xaa12 is IIe;
Xaa13 is Ala;
Xaa14 is Leu;
Xaa17 is IIe;
Xaa18 is His;
Xaa19 is Gln;
Xaa20 is Gln or Lys;
Xaa21 is Asp;
Xaa24 is Asn or Gln;
Xaa27 is Leu;
Xaa28 is Ala;
Xaa29 is Gln or Gly;
Xaa30 is Gly or Lys;
Xaa31 is Gly or Pro;
Xaa32 is Lys or Lys*;
Xaa33 is Lys or Lys*;
Xaa34 is Asn; or Xaa34 is absent;
Xaa35 is Asp; or Xaa35 is absent;
Xaa36 is Trp; or Xaa36 is absent;
Xaa37 is Lys; or Xaa37 is absent;
Xaa38 is His; or Xaa38 is absent;
Xaa39 is Asn; or Xaa39 is absent;
Xaa40 is IIe; or Xaa40 is absent;
Xaa41 is Thr; or Xaa41 is absent;
Xaa42 is Gln, or Xaa42 is absent;
Xaa43 is Lys*; or Xaa43 is absent;
Z is (i):

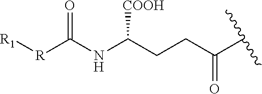

wherein R is a C₁₈ alkylene chain and R¹ is —CO₂H;
Xaa44 is absent;
Xaa45 is absent;
Xaa46 is Ser, or Xaa46 is absent; and
Xaa47 is Lys, or Xaa47 is absent.

For example, in such an embodiment:
Xaa3 is Glu;
Xaa20 is Gln;
Xaa24 is Asn;
Xaa29 is Gln;
Xaa30 is Lys;
Xaa31 is Gly;
Xaa32 is Lys*;
Xaa33 is Lys;
Xaa34 is Asn;
Xaa35 is Asp;
Xaa36 is Trp;
Xaa37 is Lys;
Xaa38 is His;
Xaa39 is Asn;
Xaa40 is IIe;
Xaa41 is Thr;
Xaa42 is Gln,
Xaa43 is absent;
Xaa46 is absent; and
Xaa47 is Lys or absent.

In such an embodiment, it is even more preferred that Xaa47 is Lys.

In another embodiment of the invention, it is preferred that:
Xaa1 is His, Phe or Tyr;
Xaa2 is AIB;
Xaa3 is Glu, Gln or His;
Xaa7 is IIe or Ser;
Xaa10 is Tyr or His;
Xaa12 is IIe or Lys;
Xaa13 is Ala, Asp, Asn, Gln, Ser, Thr or Val;
Xaa14 is Leu or Met;
Xaa17 is Gln or IIe;
Xaa18 is Ala or His;
Xaa19 is Ala or Gln;
Xaa20 is AIB, Gln, His or Lys;
Xaa21 is Asp or Glu;
Xaa24 is Asn, Gln or Glu;
Xaa25 is Trp;
Xaa27 is Leu;
Xaa28 is Ala or Asn;
Xaa29 is Gln, Gly or Thr;
Xaa30 is Gly, Lys or Lys*;
Xaa31 is Arg, Gly or Pro;
Xaa32 is Asn, Lys or Lys*;
Xaa33 is Lys or Lys*; or Xaa33 is absent;
Xaa34 is Asn or Lys*; or Xaa34 is absent;
Xaa35 is Asp; or Xaa35 is absent;
Xaa36 is Trp; or Xaa36 is absent;
Xaa37 is Lys; or Xaa37 is absent;
Xaa38 is His; or Xaa38 is absent;
Xaa39 is Asn; or Xaa39 is absent;
Xaa40 is IIe; or Xaa40 is absent;
Xaa41 is Thr; or Xaa41 is absent;
Xaa42 is Gln, or Xaa42 is absent;
Xaa43 is Lys*; or Xaa43 is absent;
Z is (i):

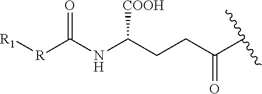

wherein R is a C₁₆ or C₁₈ alkylene chain and R¹ is —CO₂H;
Xaa44 is Thr, or Xaa44 is absent;
Xaa45 is Gly, or Xaa45 is absent;
Xaa46 is Asn, Gly or Ser, or Xaa46 is absent; and
Xaa47 is Gly or Lys, or Xaa47 is absent.

For example, in such an embodiment:
Xaa1 is Tyr;
Xaa2 is AIB;
Xaa3 is Glu;
Xaa7 is IIe or Ser;
Xaa10 is Tyr or His;
Xaa12 is IIe or Lys;
Xaa13 is Asp, Asn, Ala or Ser;
Xaa14 is Leu;
Xaa17 is IIe;
Xaa18 is His;
Xaa19 is Gln;
Xaa20 is Gln;
Xaa21 is Asp;
Xaa24 is Asn;
Xaa25 is Trp;
Xaa27 is Leu;
Xaa28 is Ala;
Xaa29 is Gln;
Xaa30 is Lys;
Xaa31 is Gly;
Xaa32 is Lys;
Xaa33 is Lys* or absent;
Xaa34 is Lys* or absent;
Xaa35 is absent;
Xaa36 is absent;
Xaa37 is absent;
Xaa38 is absent;
Xaa39 is absent;
Xaa40 is absent;
Xaa41 is absent;
Xaa42 is absent;
Xaa43 is absent;
Z is (i):

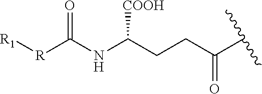

wherein R is a C₁₈ alkylene chain and R¹ is —CO₂H;
Xaa44 is absent;
Xaa45 is absent;
Xaa46 is Asn or absent; and
Xaa47 is Lys or His, or absent. For example, Xaa47 is Lys or His.

As a further example:
Xaa7 is IIe;
Xaa10 is Tyr;
Xaa12 is IIe or Lys;
Xaa13 is Ala or Asp;
Xaa33 is Lys*;
Xaa34 is absent;
Xaa46 is absent; and
Xaa47 is Lys or absent. For example, Xaa47 is Lys.

In an embodiment of the first aspect of the invention there is provided a compound of formula (I) or formula (II):

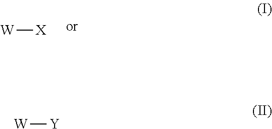

wherein
W is an amino acid sequence:

[SEQ ID NO: 7]
Xaa1-Xaa2-Xaa3-Gly4-Thr5-Phe6-Xaa7-Ser8-Asp9-Xaa10-Ser11-Xaa12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-

wherein:
Xaa1 is DTyr, His, Phe or Tyr, or absent;
Xaa2 is AIB or Ala, or absent;
Xaa3 is Asp, Gln, Glu or His;
Xaa7 is IIe, Thr or Ser;
Xaa10 is Tyr or His;
Xaa12 is IIe or Lys;
Xaa13 is AIB, Ala, Gln, Glu, Gly, His, Pro, Ser, Thr, Tyr, Val, Asp or Asn;
Xaa14 is Leu, Met or NLeu;
Xaa17 is Gln or IIe;
Xaa18 is Ala, Arg or His;
Xaa19 is Ala or Gln;
Xaa20 is AIB, Gln, His or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn; and
Xaa29 is Gln, Gly or Thr;
X, when present, is an amino acid sequence:

[SEQ ID NO: 8]
-Xaa30-Xaa31-Xaa32-Xaa33-Asn34-Asp35-Xaa36-Xaa37-His38-Xaa39-Xaa40-
Thr41-Gln42-Xaa43

wherein:
Xaa30 is Gly, Lys or Lys*;
Xaa31 is Gly or Pro;
Xaa32 is Lys or Lys*;
Xaa33 is Lys or Lys*;
Xaa36 is Trp or Lys*;
Xaa37 is Lys or Lys*;
Xaa39 is Asn or Lys*;
Xaa40 is IIe, Leu or Lys*; and
Xaa43 is Lys* or absent;
   Y, when present, is an amino acid sequence:

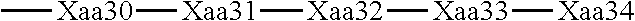

wherein:
Xaa30 is Gly or Lys, or absent;
Xaa31 is Arg, Gly or Pro, or absent;
Xaa32 is Asn or Lys, or absent;
Xaa33 is Lys, Lys* or absent;
Xaa34 is Lys* or absent;
   wherein Lys* is Lys substituted at its ε-amino group with a group of the formula:

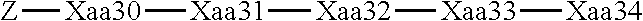

wherein:
Xaa44 is Gly, Ser or Thr, or absent;
Xaa45 is Gly or Ser, or absent;
Xaa46 is Asn, Gln, Gly, Ser or Thr, or absent;
Xaa47 is Asn, Gln, Glu, Gly, His, Lys, Pro, Ser or Thr, or absent;
   and Z is a group of the formula:

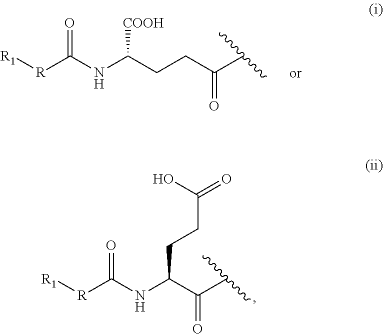

wherein R is a C₈-C₂₈ alkylene or alkenylene chain and R¹ is —CO₂H;
and wherein the compound of formula (I) or formula (II) contains one Lys* residue;
or a derivative of the compound; or a salt or solvate of the compound or the derivative.

In certain embodiments of the invention, the compound is a compound of formula (I):

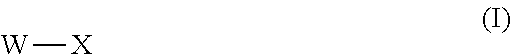

wherein Wis an amino acid sequence:

[SEQ ID NO: 9]
Xaa1-Xaa2-Xaa3-Gly4-Thr5-Phe6-Xaa7-Ser8-Asp9-Tyr10-Ser11-Ile12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-

wherein:
Xaa1 is DTyr, His, Phe or Tyr, or absent;
Xaa2 is AIB or Ala, or absent;
Xaa3 is Asp, Gln, Glu or His;
Xaa7 is IIe or Thr;
Xaa13 is AIB, Ala, Gln, Glu, Gly, His, Pro, Ser, Thr, Tyr or Val;
Xaa14 is Leu, Met or NLeu;
Xaa17 is Gln or IIe;
Xaa18 is Ala, Arg or His;
Xaa19 is Ala or Gln;
Xaa20 is AIB, Gln, His or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn; and
Xaa29 is Gln, Gly or Thr;
and X is an amino acid sequence:

[SEQ ID NO: 8]
-Xaa30-Xaa31-Xaa32-Xaa33-Asn34-Asp35-Xaa36-Xaa37-His38-Xaa39-Xaa40-
Thr41-Gln42-Xaa43

wherein:
Xaa30 is Gly, Lys or Lys*;
Xaa31 is Gly or Pro;
Xaa32 is Lys or Lys*;
Xaa33 is Lys or Lys*;
Xaa36 is Trp or Lys*;
Xaa37 is Lys or Lys*;
Xaa39 is Asn or Lys*;
Xaa40 is IIe, Leu or Lys*; and
Xaa43 is Lys* or absent.

In a preferred embodiment, when the compound of the invention is a compound of formula (I),
Xaa1 is His, Phe or Tyr;
Xaa2 is AIB;
Xaa3 is Gln, Glu or His;
Xaa7 is IIe;
Xaa13 is Ala, Gln, Thr or Val;
Xaa14 is Leu or Met;
Xaa17 is Gln or IIe;
Xaa18 is Ala or His;
Xaa19 is Ala or Gln;
Xaa20 is AIB, Gln, His or Lys;
Xaa21 is Asp or Glu;
Xaa24 is Asn, Gln or Glu;
Xaa25 is Trp;
Xaa27 is Leu;
Xaa28 is Ala or Asn; and
Xaa29 is Gln, Gly or Thr.

In such an embodiment, it is even more preferred that:
Xaa1 is Tyr;
Xaa2 is AIB;
Xaa3 is Glu or Gln; and preferably Glu;
Xaa7 is IIe;
Xaa13 is Ala;
Xaa14 is Leu;
Xaa17 is IIe;
Xaa18 is His;
Xaa19 is Gln;
Xaa20 is Gln or Lys; and preferably Gln;
Xaa21 is Asp;
Xaa24 is Asn or Gln; and preferably Asn;
Xaa25 is Trp;
Xaa27 is Leu;
Xaa28 is Ala; and
Xaa29 is Gln or Gly; and preferably Gln.

In a preferred embodiment, when the compound of the invention is a compound of formula (I),
Xaa30 is Lys or Lys*;
Xaa31 is Gly;
Xaa32 is Lys or Lys*;
Xaa33 is Lys or Lys*;
Xaa36 is Trp or Lys*;
Xaa37 is Lys or Lys*;
Xaa39 is Asn or Lys*;
Xaa40 is IIe or Lys*; and
Xaa43 is Lys* or absent.

In such an embodiment, it is even more preferred that:
Xaa30 is Lys;
Xaa32 is Lys*;
Xaa33 is Lys;
Xaa36 is Trp;
Xaa37 is Lys;
Xaa39 is Asn;
Xaa40 is IIe; and
Xaa43 is absent.

In certain embodiments of the invention, the compound is a compound of formula (II):

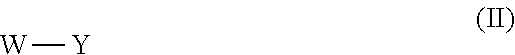

wherein W is an amino acid sequence:

[SEQ ID NO: 9]
Xaa1-Xaa2-Xaa3-Gly4-Thr5-Phe6-Xaa7-Ser8-Asp9-Tyr10-Ser11-Ile12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-

wherein:
Xaa1 is DTyr, His, Phe or Tyr, or absent;
Xaa2 is AIB or Ala, or absent;
Xaa3 is Asp, Gln, Glu or His;
Xaa7 is IIe or Thr;
Xaa13 is AIB, Ala, Gln, Glu, Gly, His, Pro, Ser, Thr, Tyr or Val;
Xaa14 is Leu, Met or NLeu;
Xaa17 is Gln or IIe;
Xaa18 is Ala, Arg or His;
Xaa19 is Ala or Gln;
Xaa20 is AIB, Gln, His or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn; and
Xaa29 is Gln, Gly or Thr;
   and Y is an amino acid sequence:

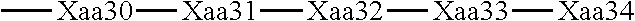

wherein:
Xaa30 is Gly or Lys, or absent;
Xaa31 is Arg, Gly or Pro, or absent;
Xaa32 is Asn or Lys, or absent;
Xaa33 is Lys or absent; and
Xaa34 is Lys*.

In another embodiment, when the compound of the invention is a compound of formula (II),
Xaa1 is Tyr;
Xaa2 is AIB;
Xaa3 is Glu;
Xaa7 is IIe, Ser or Thr;
Xaa10 is Tyr or His;
Xaa12 is IIe or Lys;
Xaa13 is AIB, Ala, Asn, Asp, Ser or Tyr;
Xaa14 is Leu;
Xaa17 is Gln or IIe;
Xaa18 is Ala or His;
Xaa19 is Ala or Gln;
Xaa20 is AIB, Gln or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn;
Xaa29 is Gln, Gly or Thr;
Xaa30 is Gly or Lys, or absent;
Xaa31 is Arg, Gly or Pro, or absent;
Xaa32 is Asn or Lys, or absent;
Xaa33 is Lys, or absent; and
Xaa34 is Lys*

In a preferred embodiment, when the compound of the invention is a compound of formula (I),
Xaa1 is His, Phe or Tyr;
Xaa2 is AIB;
Xaa3 is Gln, Glu or His;
Xaa7 is IIe;
Xaa13 is Ala, Gln, Thr or Val;
Xaa14 is Leu or Met;
Xaa17 is Gln or IIe;
Xaa18 is Ala or His;
Xaa19 is Ala or Gln;
Xaa20 is AIB, Gln, His or Lys;
Xaa21 is Asp or Glu;
Xaa24 is Asn, Gln or Glu;
Xaa25 is Trp;
Xaa27 is Leu;
Xaa28 is Ala or Asn; and
Xaa29 is Gln, Gly or Thr.

In such an embodiment, it is even more preferred that:
Xaa1 is Tyr;
Xaa2 is AIB;
Xaa3 is Glu or Gln; and preferably Glu;
Xaa7 is IIe;
Xaa13 is Ala;
Xaa14 is Leu;
Xaa17 is IIe;
Xaa18 is His;
Xaa19 is Gln;
Xaa20 is Gln or Lys; and preferably Gln;
Xaa21 is Asp;
Xaa24 is Asn or Gln; and preferably Asn;
Xaa25 is Trp;
Xaa27 is Leu;
Xaa28 is Ala; and
Xaa29 is Gln or Gly; and preferably Gln.

In a preferred embodiment, when the compound of the invention is a compound of formula (II),
Xaa30 is Lys or Gly;
Xaa31 is Gly or Pro;
Xaa32 is Lys or absent;
Xaa33 is absent; and
Xaa34 is Lys*

In such an embodiment, it is even more preferred that:
Xaa30 is Lys;
Xaa31 is Gly;
Xaa32 is absent;
Xaa33 is absent; and
Xaa34 is Lys*.

In a preferred embodiment, when the compound of the invention is a compound of formula (II),
Xaa1 is Tyr;
Xaa2 is AIB;
Xaa3 is Glu;
Xaa7 is IIe or Ser;
Xaa10 is Tyr or His;
Xaa12 is IIe or Lys;
Xaa13 is Asp, Asn, Ala or Ser;
Xaa14 is Leu;
Xaa17 is IIe;
Xaa18 is His;
Xaa19 is Gln;
Xaa20 is Gln;
Xaa21 is Asp;
Xaa24 is Asn;
Xaa25 is Trp;
Xaa27 is Leu;
Xaa28 is Ala;
Xaa29 is Gln;
Xaa30 is Lys;
Xaa31 is Gly;
Xaa32 is Lys;
Xaa33 is Lys or absent; and
Xaa34 is Lys*;
   wherein Lys* is Lys substituted at its ε-amino group with a group of the formula:

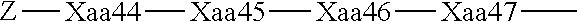

### Wherein:

Xaa44 is absent;
Xaa45 is absent;
Xaa46 is Asn or absent; and
Xaa47 is Lys or His;

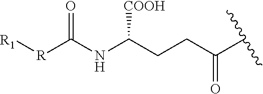

### Z is (i):

wherein R is a C₁₈ alkylene chain and R¹ is —CO₂H;

For example,
Xaa10 is Tyr;
Xaa12 is IIe or Lys;
Xaa13 is Ala or Asp;
Xaa33 is absent;
Xaa34 is Lys*;
Xaa46 is absent; and
Xaa47 is Lys.

In especially preferred embodiments, the compound is one of the compounds of the invention set out in the Table of FIG. 1.

### Derivatives and Salts

The present invention provides compounds of formula (I), derivatives of such compounds, and salts or solvates of such compounds and derivatives.

The compounds, derivatives and salts may be produced by recombinant methods which are well-known in the art or alternatively they may be produced by synthetic methods, again which are well-known in the art.

### Derivatives

Whilst in some embodiments, the invention relates to a compound of formula (I) and is not a derivative, in other embodiments the invention relates to a derivative of a compound of formula (I). The derivative may for example comprise one or more derivatisations selected from amidation, glycosylation, carbamylation, acylation, sulfation, phosphorylation, cyclization, lipidization, pegylation and fusion to another peptide or protein to form a fusion protein, for example the derivative may comprise one or more derivatisations selected from amidation, glycosylation, carbamylation, acylation, sulfation, phosphorylation, cyclization, lipidization and pegylation. The structure may be modified at random positions within the molecule, or at predetermined positions within the molecule and may include one, two, three or more attached chemical moieties.

In certain embodiments it is preferred that the primary peptide chain of compounds of the invention may be amidated at their C-terminal. Such a modification is very common in nature with approximately half of naturally occurring peptides, including certain gastrointestinal peptide hormones, being susceptible to amidation at their C-terminal. The present invention encompasses all of the generic and specific sequences disclosed herein, including in the sequence listing and drawings, in both amidated and non-amidated forms, the amidation, where present being especially preferred on the C-terminal of the primary peptide sequence.

The derivative may for example be a fusion protein, whereby the structure of formula (I) is fused to another protein or polypeptide (the fusion partner) using recombinant methods known in the art. Alternatively, such a fusion protein may be synthetically synthesized by any known method. Such a fusion protein comprises the structure of formula (I). Any suitable peptide or protein can be used as the fusion partner (e.g., serum albumin, carbonic anhydrase, glutathione-S-transferase or thioredoxin, etc.).

Such fusion proteins may be made by linking the carboxy-terminus of the fusion partner to the amino-terminus of the structure of formula (I) or vice versa. Optionally, a cleavable linker may be used to link the structure of formula (I) to the fusion partner. A resulting cleavable fusion protein may be cleaved in vivo such that an active form of a compound of the invention is released. Examples of such cleavable linkers include, but are not limited to, the linkers Asp-Asp-Asp-Asp-Tyr [SEQ ID NO: 10], Gly-Pro-Arg, Ala-Gly-Gly and His-Pro-Phe-His-Leu [SEQ ID NO: 11], which can be cleaved by enterokinase, thrombin, ubiquitin cleaving enzyme and renin, respectively. For details, see for example U.S. Pat. No. 6,410,707, the contents of which are incorporated herein by reference.

A derivative of the invention may for example be a physiologically functional derivative of the structure of formula (I). The term “physiologically functional derivative” is used herein to denote a chemical derivative of a compound of formula (I) having the same physiological function as the corresponding unmodified compound. For example, a physiologically functionally derivative may be convertible in the body to a compound of formula (I). According to the present invention, examples of physiologically functional derivatives include esters, amides, and carbamates; preferably esters and amides. In addition to the derivatisation at Lys42, compounds of the invention can be further derivatised at additional positions. For example, pharmaceutically acceptable esters and amides of the compounds of the invention may comprise a C₁₋₂₀ alkyl-, C₂₋₂₀ alkenyl-, C₅₋₁₀ aryl-, C₅₋₁₀ ar-C₁₋₂₀ alkyl-, or amino acid-ester group or amide group attached at an appropriate site, for example formed by reaction of an alkyl, alkenyl aryl, aralkyl or amino alkyl group containing an alcohol or amino moiety with an acid moiety present in the compound of formula (I), or formed by reaction of an alkyl, alkenyl aryl, aralkyl or amino alkyl group containing an activated acyl group with an alcohol or amine group present in the compound of formula (I). Examples of suitable moieties are hydrophobic substituents with 4 to 26 carbon atoms, preferably 5 to 19 carbon atoms. Suitable lipid groups include fatty acids (e.g. lauroyl (C₁₂H₂₃), palmityl (C15H₃₁), oleyl (C15H₂₉) or stearyl (C17H₃₅)) and bile acids (e.g. cholate or deoxycholate).

Methods for lipidization of sulfhydryl-containing compounds with fatty acid derivatives are disclosed in U.S. Pat. Nos. 5,936,092; 6,093,692; and 6,225,445, the contents of which are incorporated herein by reference. Fatty acid derivatives of a compound of the invention comprising a compound of the invention linked to fatty acid via a disulfide linkage may be used for delivery of a compound of the invention to neuronal cells and tissues. Lipidisation markedly increases the absorption of the compounds relative to the rate of absorption of the corresponding unlipidised compounds, as well as prolonging blood and tissue retention of the compounds. Moreover, the disulfide linkage in a lipidised derivative is relatively labile in the cells and thus facilitates intracellular release of the molecule from the fatty acid moieties. Suitable lipid-containing moieties are hydrophobic substituents with 4 to 26 carbon atoms, preferably 5 to 19 carbon atoms. Suitable lipid groups include fatty acids (e.g. lauroyl (C₁₂H₂₃), palmityl (C₁₅H₃₁), oleyl (C₁₅H₂₉) or stearyl (C₁₇H₃₅)) and bile acids (e.g. cholate or deoxycholate). Whilst lipid functionalised compounds of the invention may have benefits in certain situations, it is expected that in most cases, it will be simplest and preferred if a compound of the invention is not further derivatised, such that there are not additional lipid groups present.

Cyclization methods include cyclization through the formation of a disulfide bridge, and head-to-tail cyclization using a cyclization resin. Cyclized peptides may have enhanced stability, including increased resistance to enzymatic degradation, as a result of their conformational constraints. Cyclization may in particular be expedient where the uncyclized peptide includes an N-terminal cysteine group. Suitable cyclized peptides include monomeric and dimeric head-to-tail cyclized structures. Cyclized peptides may include one or more additional residues, especially an additional cysteine incorporated for the purpose of formation of a disulfide bond or a side chain incorporated for the purpose of resin-based cyclization.

The derivative may for example be a PEGylated structure of formula (I). Derivatives which are PEGylated compounds of the invention may provide additional advantages such as increased solubility, stability and circulating time of the polypeptide, or decreased immunogenicity (see U.S. Pat. No. 4,179,337, the contents of which are incorporated herein by reference).

Chemical moieties for derivatisation of a compound of the invention may also be selected from water soluble polymers such as polyethylene glycol, ethylene glycol/propylene glycol copolymers, carboxymethylcellulose, dextran, polyvinyl alcohol and the like. A polymer moiety for derivatisation of a compound of the invention may be of any molecular weight and may be branched or unbranched. For ease in handling and manufacturing, the preferred molecular weight of a polyethylene glycol for derivatisation of a compound of the invention is from about 1 kDa to about 100 kDa, the term “about” indicating that in preparations of polyethylene glycol, some molecules will weigh more, some less, than the stated molecular weight. Polymers of other molecular weights may be used, depending on the desired therapeutic profile, for example the duration of sustained release desired, the effects, if any, on biological activity, the ease in handling, the degree or lack of antigenicity and other known effects of the polyethylene glycol to a therapeutic protein or analog. For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

### Salts

Salt forms of compounds of formula (I) and of derivatives of such compounds also form part of the invention. In some embodiments the salt is a salt of a compound of formula (I). In other embodiments the salt is a salt of a derivative of a compound of formula (I).

Salts of compounds of the invention include those which are pharmaceutically acceptable, i.e. which are suitable for use in medicine. However, salts having non-pharmaceutically acceptable counterions are also within the scope of the present invention, for example, for use as intermediates in the preparation of the compounds.

Suitable salts according to the invention include those formed with organic or inorganic acids or bases. Pharmaceutically acceptable acid addition salts include those formed with hydrochloric, hydrobromic, sulphuric, nitric, citric, tartaric, acetic, phosphoric, lactic, pyruvic, acetic, trifluoroacetic, succinic, perchloric, fumaric, maleic, glycolic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic acid may be useful as intermediates in obtaining the compounds of the invention in final form.

Pharmaceutically acceptable salts with bases include ammonium salts, alkali metal salts, for example potassium and sodium salts, alkaline earth metal salts, for example calcium and magnesium salts, and salts with organic bases, for example dicyclohexylamine and N-methyl-D-glucomine.

### Solvates

Those skilled in the art of organic and/or medicinal chemistry will appreciate that many organic compounds can form complexes with solvents in which they are reacted or from which they are precipitated or crystallized. Such complexes are known as “solvates”. For example, a complex with water is known as a “hydrate”. The invention also encompasses solvates of the compounds of formula (I), solvates of derivatives of the compounds, and solvates of salts of the derivatives.

Those skilled in the art of organic and/or medicinal chemistry will also appreciate than many organic compounds can exist in different forms, including as amorphous material and/or in one or more crystalline forms. Different physical forms of organic compounds are known as polymorphs. The invention also encompasses all such different physical forms of the compounds of formula (I), as well as different physical forms of their derivatives and salts.

### Biological Activity

Compounds of the invention have activity at the human GIP receptor and can be considered GIP receptor agonists. This may be assessed by, for example, an in vitro or cellular binding assay or by a reporter assay. Preferred compounds of the invention exhibit an activity at the human GIP receptor that is at least 1/50^{th} that of human GIP, preferably an activity which is at least 1/30^{th}, 1/20^{th}, 1/10^{th}, 1/5th, 1/3rd or 1/2 that of human GIP, for example when tested in accordance with the assay described in the examples section below.

Methods of assessing activity at the GIP receptor are well known. For example, Homogeneous Time Resolved Fluorescence (R. J. Samms et al., GIPR agonism mediates weight-independent insulin sensitization by tirzepatide in obese mice, J. Clin. Invest., 2021, 131 (12): e146353) detects changes in cAMP accumulation in response to Gs-coupled G protein-coupled receptor (GPCR) activation. A specific method is described herein below.

Preferred compounds of the invention are effective in promoting insulin release/secretion. This may be assessed by, for example, an in vitro or in vivo assay. Methods of assessing release of insulin are well known.

Compounds of the invention fulfil some, or more preferably all, of the following criteria:
1) Sustained bioactivity at the human GIP receptor resulting in inhibition of appetite;
2) Activity in promoting insulin release;
3) High solubility in aqueous solution at pH 3.0-8.0 to allow an effective dose to be administered in a low volume injection (thereby resulting in lower pain of injection). Solubility may be easily assessed by simple in vitro tests;
4) Long period of activity in vivo (as assessed in humans or an animal model) so as to permit injections no more frequently than daily and preferably no more than twice, or more preferably no more than once a week, whilst still producing acceptable therapeutic or cosmetic benefits;
5) Good weight loss (as assessed in human subjects or an animal model, where assessment may be completed with co-administration of a GLP1r agonists).

According to certain embodiments of the invention, especially embodiments relating to weight loss, obesity, carbohydrate metabolism and diabetes, the compounds, derivatives and salts of the invention have one, several or all of the following features:
A. Sufficient solubility between pH 3.0 and pH 8.0 to permit an effective dose to be administered in a volume of less than 1 mL, less than 0.5 mL or less than 0.3 mL;
B. Activation of cAMP signalling in cells over-expressing the human GIP Receptor;
C. One, several or all of the further 1 to 5 features listed above.

### Pharmacokinetics, Duration of Action and Solubility

Compounds of the present invention exhibit potent and prolonged duration of action in vivo following subcutaneous administration. In order to achieve this, the compounds are required to have both good activity at the biological target, and excellent pharmacokinetic properties.

Compounds of the present invention have a therapeutically useful duration of action and that manifests itself in the beneficial effects being observed in the experiments described herein below over several days. The half-life of compounds of the invention was assessed in a pig PK model. Preferred compounds of the invention were found to have a half-life equivalent to or longer than semaglutide. As well as exhibiting a long in vivo half-life, the compounds of the invention have good storage stability, with no significant degradation seen on storage in solution for 4 weeks at 4° C.

Poor water solubility is a known problem for lipid containing molecules. In contrast, the compounds of the invention have very good solubility.

### Conditions

The invention also provides a compound, derivative or salt of the invention, or a composition comprising the compound, derivative or salt together with a pharmaceutically acceptable carrier and optionally a further therapeutic agent, for use as a medicament.

The invention also provides a method of treating or preventing a disease or disorder or other non-desired physiological state in a subject comprising administration of a therapeutically effective amount of a compound, derivative or salt of the invention, or of a composition comprising the compound, derivative or salt together with a pharmaceutically acceptable carrier and optionally a further therapeutic agent. Preferably the compound, derivative, salt or composition is administered subcutaneously.

According to certain embodiments, the disease or disorder or other non-desired physiological state is diabetes or obesity, and particularly diabetes (e.g. type II diabetes).

According to certain embodiments, the disease or disorder or other non-desired physiological state may be the physiological state of being overweight.

The subject to whom the compound is administered may be overweight, for example, obese. Alternatively, or in addition, the subject may be diabetic, for example having insulin resistance or glucose intolerance, or both. The subject may have diabetes mellitus, for example, the subject may have Type II diabetes. The subject may be overweight, for example, obese and have diabetes mellitus, for example, Type II diabetes.

In addition, or alternatively, the subject may have, or may be at risk of having, a disorder in which obesity or being overweight is a risk factor. Such disorders include, but are not limited to, heart disease, cardiovascular disease, for example hypertension, atherosclerosis, congestive heart failure, and dyslipidemia; stroke; gallbladder disease; osteoarthritis; sleep apnea; reproductive disorders for example, polycystic ovarian syndrome; cancers, for example breast, prostate, colon, endometrial, kidney, and esophagus cancer; varicose veins; acanthosis nigricans; eczema; exercise intolerance; insulin resistance; hypertension hypercholesterolemia; cholithiasis; osteoarthritis; orthopedic injury; insulin resistance, for example, type 2 diabetes and syndrome X; and thromboembolic disease (see Kopelman, Nature 404:635-43, 2000; Rissanen et al., British Med. J. 301, 835, 1990).

Other disorders associated with obesity include depression, anxiety, panic attacks, migraine headaches, PMS, chronic pain states, fibromyalgia, insomnia, impulsivity, obsessive compulsive disorder, and myoclonus. Certain neurological disorders and certain firms of neurological degeneration are also associated with obesity. Furthermore, obesity is a recognized risk factor for increased incidence of complications of general anesthesia (see e. g., Kopelman, Nature 404:635-43, 2000). In general, obesity reduces life span and carries a serious risk of co-morbidities such as those listed above.

Other diseases or disorders associated with obesity are birth defects, maternal obesity being associated with increased incidence of neural tube defects, carpal tunnel syndrome (CTS); chronic venous insufficiency (CVI); daytime sleepiness; deep vein thrombosis (DVT); end stage renal disease (ESRD); gout; heat disorders; impaired immune response; impaired respiratory function; infertility; liver disease; lower back pain; obstetric and gynecologic complications; pancreatitis; as well as abdominal hernias; acanthosis nigricans; endocrine abnormalities; chronic hypoxia and hypercapnia; dermatological effects; elephantitis; gastroesophageal reflux; heel spurs; lower extremity edema; mammegaly which causes considerable problems such as bra strap pain, skin damage, cervical pain, chronic odours and infections in the skin folds under the breasts, etc.; large anterior abdominal wall masses, for example abdominal panniculitis with frequent panniculitis, impeding walking, causing frequent infections, odours, clothing difficulties, low back pain; musculoskeletal disease; pseudotumor cerebri (or benign intracranial hypertension), and sliding hiatal hernia.

In some embodiments, the disease or disorder may be non-alcoholic fatty liver disease.

According to certain embodiments the disease or disorder or other non-desired physiological state may be being of a non-desired weight despite not being obese or overweight. The subject may be of normal weight (this includes but is not limited to subjects who were previously overweight or obese and who wish to prevent a return to an unhealthy weight). A subject may be a subject who desires weight loss, for example female and/or male subjects who desire a change in their appearance. In some cases where the subject is of a normal weight, aspects of the invention may relate to cosmetic treatment rather than to therapeutic treatment.

The invention also provides a method of reducing appetite in a subject, reducing food intake in a subject, reducing calorie intake in a subject, improving insulin release in a subject, improving carbohydrate metabolism in a subject, and/or improving carbohydrate tolerance in a subject, comprising administration of a therapeutically effective amount of a compound, derivative, salt or composition of the invention. Such methods may relate to treating subjects having a pre-diabetic state such as insulin insensitivity or pre-diabetes.

The invention also provides a method for improving a lipid profile in a subject comprising administration of a therapeutically effective amount of a compound, derivative, salt or composition of the invention. The invention also provides a method for alleviating a condition or disorder that can be alleviated by reducing nutrient availability comprising administration of a therapeutically effective amount of a compound, derivative, salt or composition of the invention.

A compound, derivative, salt or composition of the invention may be used for weight control and treatment, for example reduction or prevention of obesity, in particular any one or more of the following: preventing and reducing weight gain; inducing and promoting weight loss; and reducing obesity as measured by the Body Mass Index. A compound, derivative, salt or composition of the invention may be used in maintaining any one or more of a desired body weight, a desired Body Mass Index, a desired appearance and good health.

The present invention may also be used in treating, prevention, ameliorating or alleviating conditions or disorders caused by, complicated by, or aggravated by a relatively high nutrient availability. The term “condition or disorder which can be alleviated by reducing caloric (or nutrient) availability” is used herein to denote any condition or disorder in a subject that is either caused by, complicated by, or aggravated by a relatively high nutrient availability, or that can be alleviated by reducing nutrient availability, for example by decreasing food intake. Subjects who are insulin resistant, glucose intolerant, or have any form of diabetes mellitus, for example, type 1, 2 or gestational diabetes, can also benefit from methods in accordance with the present invention.

Conditions or disorders associated with increased caloric intake include, but are not limited to, insulin resistance, glucose intolerance, obesity, diabetes, including type 2 diabetes, eating disorders, insulin-resistance syndromes, and Alzheimer's disease. J. Cereb. Blood Flow Metab. 2011 Apr. 13 (Teramoto S et al.) discusses the use of both GLP-1 and exendin-4 to confer cardioprotection after myocardial infarction and demonstrates that exendin-4 may be used to provide neuroprotection against cerebral ischemia-reperfusion injury. The study showed that mice receiving a transvenous injection of exendin-4, after a 60-minute focal cerebral ischemia showed significantly reduced infarct volume and improved functional deficit as well as suppressed oxidative stress, inflammatory response, and cell death after reperfusion. The study provided evidence that the protective effect of exendin-4 is mediated through increased intracellular CAMP levels and suggested that exendin-4 is potentially useful in the treatment of acute ischemic stroke.

Accordingly, the invention also provides a method of providing cytoprotection in a subject, such as providing cardiac protection, providing neuroprotection and/or treating or preventing neurodegeneration, comprising administration of a therapeutically effective amount of a compound, derivative, salt or composition of the invention. In certain embodiments the disease or disorder or other non-desired physiological state which the compound, derivative, salt or composition of the invention may be used to treat or prevent is neurodegeneration. Such neurodegeneration may be caused by apoptosis, necrosis or loss of function of neuronal cells, preferably in the CNS. Neurodegeneration treated or prevented may be that following a brain injury (for example following physical trauma or following a non-traumatic injury such a stroke, tumor, hypoxia, poisoning, infection, ischemia, encephalopathy or substance abuse).

Alternatively or additionally, neurodegeneration may be prevented or treated in a subject having (or diagnosed as having a predisposition to) a neurodegenerative disease such as Alzheimer's disease, Parkinson's disease, Gehrig's disease (Amyotrophic Lateral Sclerosis), Huntington's disease, Multiple Sclerosis, other demyelination related disorders, senile dementia, subcortical dementia, arteriosclerotic dementia, AIDS-associated dementia, other dementias, cerebral vasculitis, epilepsy, Tourette's syndrome, Guillain Barre Syndrome, Wilson's disease, Pick's disease, neuroinflammatory disorders, encephalitis, encephalomyelitis, meningitis, other central nervous system infections, prion diseases, cerebellar ataxias, cerebellar degeneration, spinocerebellar degeneration syndromes, Friedrich's ataxia, ataxia teangiectasia, spinal dysmyotrophy, progressive supranuclear palsy, dystonia, muscle spasticity, tremor, retinitis pigmentosa, striatonigral degeneration, mitochondrial encephalomyopathies, neuronal ceroid lipofuscinosis. Preferably, the neurodegenerative disease is selected from the group consisting of Alzheimer's disease, Parkinson's disease, Gehrig's disease (Amyotrophic Lateral Sclerosis) and Huntington's disease. In such circumstances the treatment would be regarded as neuroprotective. According to certain preferred embodiments, the treatment is neuroprotective following cerebral ischemia or neuroprotective in a subject having a neurodegenerative disease or diagnosed as having a predisposition to a neurodegenerative disease.

According to other embodiments the disease or disorder or other non-desired physiological state is cardiac degeneration (in particular myocardial degeneration by apoptosis, necrosis or loss of function of myocardial cells), in which case the compound, derivative, salt or composition according to the invention provides cardiac protection. According to certain preferred embodiments that treatment is protective of myocardial function following myocardiac infarction.

The invention also provides a compound, derivative, salt or composition of the invention, for use in the treatment of obesity or diabetes.

The invention also provides a compound, derivative, salt or composition of the invention, for use in increasing energy expenditure of a subject, improving insulin release in a subject, improving carbohydrate tolerance in a subject and/or improving carbohydrate metabolism in a subject. Such use may relate to treating subjects having a pre-diabetic state such as insulin insensitivity or pre-diabetes.

The invention also provides a compound, derivative, salt or composition of the invention, for use in the reduction of appetite in a subject, use in the reduction of food intake in a subject, use in the reduction of calorie intake in a subject, use in improving insulin release in a subject, and/or use in improving carbohydrate tolerance in a subject. Such use may relate to treating subjects having a pre-diabetic state such as insulin insensitivity or pre-diabetes.

The invention also provides a compound, derivative, salt or composition of the invention, for use as a cytoprotective agent (e.g. in treating or preventing neurodegeneration, providing neuroprotection and/or providing cardiac protection). For example, the compound, derivative, salt or composition may be for use in myocardial protection in a subject following myocardial infarction, or for use in neuroprotection in a subject following cerebral ischemia or stroke, or for use in neuroprotection in a subject having a chronic neurodegenerative disease. Various features of neuroprotective or cardioprotective use of the compound, derivative, salt or composition may be as outlined above in relation to methods of the invention.

In the case of neuroprotection, the subject may have experienced previously a brain injury, stroke or other event causing cerebral ischemia. Alternatively, the subject may have or have been diagnosed with a predisposition to develop a chronic neurodegenerative disease. In the case of cardioprotection the subject may have experienced previously an event causing myocardial ischemia such as a myocardial infarction and angina. According to some embodiments a compound, derivative, salt or composition of the invention may be administered as soon as possible after the subject has experienced a suspected myocardial infarction. According to certain embodiments a compound, derivative, salt or composition of the invention may be administered as soon as possible after the subject has experienced as suspected stroke.

The invention also provides use of a compound, derivative, salt or composition of the invention for the manufacture of a medicament for the treatment of obesity or diabetes, of a subject, who may be as described above in reference to other aspects of the invention.

The invention also provides use of a compound, derivative or salt of the invention for the manufacture of a medicament for improving insulin release in a subject, for improving carbohydrate tolerance in a subject and/or improving carbohydrate metabolism in a subject. Such use may relate to treating subjects with a pre-diabetic state such as insulin insensitivity or pre-diabetes.

The invention also provides use of a compound, derivative or salt of the invention for the manufacture of a medicament for the reduction of appetite in a subject, reducing food intake in a subject, reducing calorie intake in a subject, improving insulin release in a subject, and/or use in improving carbohydrate tolerance in a subject.

The invention also provides use of a compound, derivative or salt of the invention for the manufacture of a medicament for providing cytoprotection (e.g. preventing or treating neurodegeneration, providing neuroprotection and/or providing cardiac protection) of a subject, who may be as described above in reference to other aspects of the invention.

According to certain embodiments the compound, derivative, salt or composition of the invention is to be administered parentally. According to other embodiments the compound, derivative, salt or composition of the invention is administered subcutaneously, intravenously, intramuscularly, intranasally, transdermally or sublingually. According to other embodiments the compound, derivative, salt or composition of the invention is administered orally. In one preferred embodiment compound, derivative, salt or composition of the invention is administered subcutaneously.

The compound, derivative, salt or composition of the invention is preferably used in the treatment of a human subject. However, while the compound, derivative, salt or composition of the invention will typically be used to treat human subjects they may also be used to treat similar or identical conditions in other vertebrates for example other primates; farm animals for example swine, cattle and poultry; sport animals for example horses; or companion animals for example dogs and cats.

### Compositions

It is preferable for the compound of formula (I), or the derivative and/or the salt thereof, to be present in a pharmaceutical formulation or composition. Accordingly, the invention provides a composition comprising a compound, derivative or salt of the invention together with a pharmaceutically acceptable excipient and optionally another therapeutic ingredient. Compositions comprising the compound, derivative or salt are suitable for pharmaceutical use. According to certain preferred embodiments the composition is present in a syringe or other administration device for subcutaneous administration to humans. According to certain preferred embodiments the composition has a pH of 3.0-8.0. Compositions of the invention may take the form of a pharmaceutical formulation as described below.

The pharmaceutical formulations according to the invention include those suitable for oral, parenteral (including subcutaneous, intradermal, intramuscular, intravenous, and intra-articular), inhalation (including fine particle dusts or mists which may be generated by means of various types of metered dose pressurized aerosols, nebulizers or insufflators), rectal and topical (including dermal, transdermal, transmucosal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42: 2S, 1988, the contents of which are incorporated herein by reference.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds or, particularly in the case of extended release, by the use of devices such as subcutaneous implants or osmotic pumps. The present compounds may also be administered liposomally.

Preferably, compositions according to the invention are suitable for subcutaneous administration, for example by injection. According to certain embodiments the composition may contain metal ions, for example copper, iron, aluminium, zinc, nickel or cobalt ions. The presence of such ions may limit solubility and thus delay absorption into the circulatory system from the site of subcutaneous administration.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. Such compositions may also include a permeation enhancer. The compounds of the invention may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxypropyl cellulose (HPC), hydroxypropyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono-or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include any compounds known to be deleterious to peptide compounds.

Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art. Conveniently in compositions for nasal aerosol or inhalation administration the compound of the invention is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated to contain a powder mix of the compound and a suitable powder base, for example lactose or starch. In one specific, non-limiting example, a compound of the invention is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations for rectal administration may be presented as a retention enema or a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds, derivatives and salts of the invention may also be suitably administered as sustained-release systems. Suitable examples of sustained-release systems of the invention include suitable polymeric materials, for example semi-permeable polymer matrices in the form of shaped articles, e.g., films, or microcapsules; suitable hydrophobic materials, for example as an emulsion in an acceptable oil; or ion exchange resins; and sparingly soluble derivatives of the compound of the invention, for example, a sparingly soluble salt. Sustained-release systems may be administered orally; rectally; parenterally; intracisternally; intravaginally; intraperitoneally; topically, for example as a powder, ointment, gel, drop or transdermal patch; bucally; or as an oral or nasal spray.

Preparations for administration can be suitably formulated to give controlled release of compounds, derivatives and salts of the invention. For example, the pharmaceutical compositions may be in the form of particles comprising one or more of biodegradable polymers, polysaccharide jellifying and/or bioadhesive polymers, amphiphilic polymers, agents capable of modifying the interface properties of particles of the compounds of the invention. These compositions exhibit certain biocompatibility features which allow a controlled release of the active substance, see U.S. Pat. No. 5,700,486, the contents of which are incorporated by reference.

The use of a controlled release composition is preferred for indications such as the treatment of obesity and/or diabetes, where maximising the time period between injections is desirable. However, for indications such as providing neuroprotection or cardiac protection (e.g. following suspected myocardial infarction or stroke), where it is desired to achieve a therapeutic plasma concentration of the active agent in as short a time period as possible, an immediate release formulation will be preferred. In such cases, a dosage regime comprising administration of a dose of an immediate release formulation of the active agent (i.e. as soon as possible after suspected myocardial infarction or stroke) and subsequent administration of a dose of a controlled release formulation of the active agent may be preferred.

A compound, derivative or salt of the invention may be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by a continuous subcutaneous infusion, for example, using a mini-pump. An intravenous bag solution may also be employed. The key factor in selecting an appropriate dose is the result obtained, as measured by decreases in total body weight or ratio of fat to lean mass, or by other criteria for measuring control or prevention of obesity or prevention of obesity-related conditions, as are deemed appropriate by the practitioner. Other controlled release systems are discussed in the review by Langer (Science 249:1527-1533, 1990) which is incorporated herein by reference. In another aspect of the disclosure compounds of the invention are delivered by way of an implanted pump, described, for example, in U.S. Pat. Nos. 6,436,091; 5,939,380; 5,993,414, the contents of which are incorporated herein by reference.

Implantable drug infusion devices are used to provide patients with a constant and long-term dosage or infusion of a drug or any other therapeutic agent. Essentially such device may be categorized as either active or passive. A compound, derivative or salt of the present invention may be formulated as a depot preparation. Such a long acting depot formulation can be administered by implantation, for example subcutaneously or intramuscularly; or by intramuscular injection. Thus, for example, the active ingredient can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of the active agent of the invention may be administered as a single pulse dose, as a bolus dose, or as pulse doses administered over time. Thus, in pulse doses, a bolus administration of the active agent is provided, followed by a time period wherein no active agent is administered to the subject, followed by a second bolus administration. In specific, non-limiting examples, pulse doses are administered during the course of a day, during the course of a week, or during the course of a month.

### Combination Treatments

In certain embodiments, a therapeutically effective amount of a compound, derivative, salt or composition of the invention is administered with a therapeutically effective amount of a further agent or agents. The compound, derivative or salt may for example be administered simultaneously with one or more further therapeutic agent(s), or it may be administered sequentially or separately. Accordingly, the invention provides a compound, derivative or salt of the invention for use as a medicament, wherein the compound, derivative or salt is for use with a therapeutically effective amount of a further therapeutic agent or agents (e.g. for administration simultaneously, sequentially or separately). In certain embodiments, the active agent of the invention is formulated and administered with a further therapeutic agent or agents as a single dose.

In certain embodiments, the further therapeutic agent or agents is/are an additional anti-diabetic, appetite suppressant, a food-intake-reducing, plasma glucose-lowering or plasma lipid-altering agent. Specific, non-limiting examples of an additional appetite suppressant include amfepramone (diethylpropion), phentermine, mazindol and phenylpropanolamine, fenfluramine, dexfenfluramine, phendimetrazine, benzphetamine, sibutramine, rimonabant, topiramate, fluoxetine, bupropion, zonisamide, naltrexone, orlistat and cetilistat. Specific, non-limiting examples of an additional anti-diabetic agent include metformin, phenformin, rosiglitazone, pioglitazone, troglitazone, repaglinide, nateglinide, tolbutamide, acetohexamide, tolazamide, chlorpropamide, glipizide, glyburide, glimepiride, gliclazide, fibroblast growth factor 21, miglitol, acarbose, exenatide, pramlintide, vildagliptin and sitagliptin.

In a preferred embodiment, the further therapeutic agent or agents is/are GLP-1 receptor agonists. In another preferred embodiment, the further therapeutic agent or agents is/are amylin receptor agonists. In another preferred embodiment, the further therapeutic agent or agents is/are glucagon receptor agonists.

In an alternative embodiment, the further therapeutic agents are GLP-1 receptor agonists and amylin receptor agonists. In another alternative embodiment, the further therapeutic agents are GLP-1 receptor agonists and glucagon receptor agonists. In another alternative embodiment, the further therapeutic agents are amylin receptor agonists and glucagon receptor agonists.

In a further alternative embodiment, the further therapeutic agents are GLP-1 receptor agonists, amylin receptor agonists and glucagon receptor agonists.

In alternative embodiments, the further therapeutic agent or agents is/are an additional cardioprotective or neuroprotective agent. Specific, non-limiting, examples of additional cardioprotective agents include aspirin, N-acetylcysteine, phenethylamines, coenzyme Q10, vitamin E, vitamin C, L-carnitine, carvedilol and dexrazoxane. Specific, non-limiting examples of neuroprotective agents include statins such as simvastatin, steroids such as progesterone, minocycline, resveratrol and vitamin E. Examples of agents used for the treatment of Parkinson's disease include anticholinergics, pramipexole, bromocriptine, levodopa, carbidopa, rasagiline, amantadine and ropinirole.

### Dosages

A compound, derivative, salt or composition of the invention may be administered whenever the effect, e.g., appetite suppression, decreased food intake or decreased caloric intake, is desired, or slightly before to whenever the effect is desired, such as, but not limited to, about 10 minutes, about 15 minutes, about 30 minutes, about 60 minutes, about 90 minutes, or about 120 minutes, before the time the effect is desired. The therapeutically effective amount of the active agent of the invention will be dependent on the molecule utilized, the subject being treated, the severity and type of the affliction, and the manner and route of administration. For example, a therapeutically effective amount of a compound of the invention may vary from about 0.01 μg per kilogram (kg) body weight to about 1 g per kg body weight, for example about 0.1 μg to about 20 mg per kg body weight, for example about 1 μg to about 5 mg per kg body weight, or about 5 μg to about 1 mg per kg body weight.

In one embodiment of the invention, a compound, derivative or salt of the invention may be administered to a subject at from 0.5 to 1,333 nmol per kg bodyweight, for example 1 to 1,333 nmol/kg bodyweight, for example 2 to 1,000 nmol per kg bodyweight, for example 4 to 1,333 nmol per kg bodyweight, for example from 5 to 1,000 nmol per kg bodyweight, for example at from 10 to 750 nmol per kg bodyweight, for example at from 20 to 500 nmol per kg bodyweight, in particular at from 30 to 240 nmol per kg bodyweight. In a preferred embodiment, a high activity compound of the invention is administered to a subject at from 0.2 to 10 nmol per kg bodyweight, for example 0.5 to 5.0 nmol/kg bodyweight, for example 1.0 to 2.0 nmol per kg bodyweight, for example 1.5 nmol per kg bodyweight., For a 75 kg subject, such doses correspond to dosages of from 37.5 nmol to 100 μmol, for example from 75 nmol to 100 μmol, for example from 150 nmol to 100 μmol, for example from 300 nmol to 100 μmol, for example from 375 nmol to 75 μmol, for example from 750 nmol to 56.25 μmol, for example from 1.5 to 37.5 μmol, in particular from 2.25 to 18 μmol. In a preferred embodiment for a high activity compound of the invention, for a 75 kg subject, such doses correspond to dosages of from 15 to 750 nmol, for example 37.5 to 375.0 nmol, for example 75 to 150 nmol, for example 112.5 nmol. The invention also contemplates dosages ranges bounded by any of the specific dosages mentioned herein.

The exact dose is readily determined by one of skill in the art based on the potency of the specific compound utilized, the route of delivery of the compound and the age, weight, sex and physiological condition of the subject.

For a compound with a long blood half-life, the doses discussed above may be given, for example, once or twice per month, or once, twice, three-times or four-times per week. For a preferred compound, a dose may be given no more frequently than once a week. Alternatively, for a compound with a shorter half-life in the blood, the doses discussed above may be given, for example, once, twice, three-times or four-times a day or once or twice a week. In some embodiments, a dose may be given once every 2, 3 or 4 days. According to certain embodiments they may be administered once shortly before each meal to be taken.

### Examples

The invention is further described with reference to the following non-limiting examples.

### Materials and Methods

### Peptide Synthesis

Peptide synthesis was carried out on a tricyclic amide linker resin. Amino acids were attached using the Fmoc strategy. For the portion of the molecule from Xaa1 to Xaa43, each amino acid was added sequentially from the C- to the N-termini. Peptide couplings were mediated using reagents such as HBTU. Peptide cleavage from the resin was achieved with trifluoracetic acid in the presence of scavengers. In a second stage, the lysine residue to be substituted was functionalised at its ε-amino group following deprotection of the ε-amino group. The chain on the substituted lysine residue was then constructed sequentially using the same amino acid attachment chemistry.

Peptides were purified by reverse phase HPLC. Quality control was performed on all purified peptides and peptides were shown in most cases to be greater than 90% pure by HPLC in two buffer systems. MALDI-MS showed the expected molecular ion.

### Example Synthesis

Example compound 91 (GIP Ref 117) was prepared as follows using standard Fmoc chemistry:
1. Resin preparation: 1-chloro-2-[chloro (diphenyl)methyl]benzene resin (0.3 mmol, 1.00 eq., Sub 1.08 mmol/g) and Fmoc-Gln (Trt)—OH (1.0 eq.) in DCM (20 mL) was added to DIEA (4.0 eq.) and agitated with N₂ for 2.5 h at 25° C. Then the mixture was filtered to get the resin.
2. Deprotection: 20% piperidine in DMF (25.00 mL) was added to the resin and the mixture was agitated with N₂ for 15 min at 20° C. The resin was washed with DMF (25.00 mL×5) and filtered to get the resin.
3. Coupling: A solution of HBTU (2.85 eq.) and Fmoc-Thr (tBu)—OH (3.00 eq.) in DMF (20 mL) was added with DIEA (6.00 eq.) to the resin and agitated with N₂ for 30 min at 20° C. The resin was then washed with DMF (20.0 mL×5).
4. Steps 2 and 3 were repeated using the reagents in Table 1 for the coupling of the following amino acids (3-42):

TABLE 1

| # | Materials | Coupling reagents |
|---|---|---|
| 3 | Fmoc-Ile-OH (3.00 eq) | HBTU (2.85 eq) and DIEA (6.00 eq) |
| 4 | Fmoc-Asn(Trt)-OH (3.00 eq) | HBTU (2.85 eq) and DIEA (6.00 eq) |
| 5 | Fmoc-His(Trt)-OH (3.00 eq) | HBTU (2.85 eq) and DIEA (6.00 eq) |
| 6 | Fmoc-Lys(Boc)-OH (3.00 eq) | HBTU (2.85 eq) and DIEA (6.00 eq) |
| 7 | Fmoc-Trp(Boc)-OH (3.00 eq) | HBTU (2.85 eq) and DIEA (6.00 eq) |
| 8 | Fmoc-Asp(OtBu)-OH (3.00 eq) | HBTU (2.85 eq) and DIEA (6.00 eq |
| 9 | Fmoc-Asn(Trt)-OH (3.00 eq) | HBTU (2.85 eq) and DIEA (6.00 eq) |
| 10 | Fmoc-Lys(Boc)-OH (3.00 eq) | HBTU (2.85 eq) and DIEA (6.00 eq) |
| 11 | Fmoc-Lys(Dde)-OH (3.00 eq) | HBTU (2.85 eq) and DIEA (6.00 eq) |
| 12 | Fmoc-Gly-OH (3.00 eq) | HBTU (2.85 eq) and DIEA (6.00 eq) |
| 13 | Fmoc-Lys(Boc)-OH (3.00 eq) | HBTU (2.85 eq) and DIEA (6.00 eq) |
| 14 | Fmoc-Gln(Trt)-OH (3.00 eq) | HBTU (2.85 eq) and DIEA (6.00 eq) |
| 15 | Fmoc-Ala-OH (3.00 eq) | HBTU (2.85 eq) and DIEA (6.00 eq) |
| 16 | Fmoc-Leu-OH (3.00 eq) | HBTU (2.85 eq) and DIEA (6.00 eq) |
| 17 | Fmoc-Leu-OH (3.00 eq) | HBTU (2.85 eq) and DIEA (6.00 eq) |
| 18 | Fmoc-Trp(Boc)-OH (3.00 eq) | HBTU (2.85 eq) and DIEA (6.00 eq) |
| 19 | Fmoc-Asn(Trt)-OH (3.00 eq) | HBTU (2.85 eq) and DIEA (6.00 eq) |
| 20 | Fmoc-Val-OH (3.00 eq) | HBTU (2.85 eq) and DIEA (6.00 eq) |
| 21 | Fmoc-Phe-OH (3.00 eq) | HBTU (2.85 eq) and DIEA (6.00 eq) |
| 22 | Fmoc-Asp(OtBu)-OH (3.00 eq) | HBTU (2.85 eq) and DIEA (6.00 eq) |
| 23 | Fmoc-Gln(Trt)-OH (3.00 eq) | HBTU (2.85 eq) and DIEA (6.00 eq) |
| 24 | Fmoc-Gln(Trt)-OH (3.00 eq) | HBTU (2.85 eq) and DIEA (6.00 eq) |
| 25 | Fmoc-His(Trt)-OH (3.00 eq) | HBTU (2.85 eq) and DIEA (6.00 eq) |
| 26 | Fmoc-Ile-OH (3.00 eq) | HBTU (2.85 eq) and DIEA (6.00 eq) |
| 27 | Fmoc-Lys(Boc)-OH (3.00 eq) | HBTU (2.85 eq) and DIEA (6.00 eq) |
| 28 | Fmoc-Asp(OtBu)-OH (3.00 eq) | HBTU (2.85 eq) and DIEA (6.00 eq) |
| 29 | Fmoc-Leu-OH (3.00 eq) | HBTU (2.85 eq) and DIEA (6.00 eq) |
| 30 | Fmoc-Aib-OH (3.00 eq) | HBTU (2.85 eq) and DIEA (6.00 eq) |
| 31 | Fmoc-Ile-OH (3.00 eq) | HATU (2.85 eq) and DIEA (6.00 eq) |
| 32 | Fmoc-Ser(tBu)-OH (3.00 eq) | HATU (2.85 eq) and DIEA (6.00 eq) |
| 33 | Fmoc-Tyr(tBu)-OH (3.00 eq) | HATU (2.85 eq) and DIEA (6.00 eq) |
| 34 | Fmoc-Asp(OtBu)-OH (3.00 eq) | HATU (2.85 eq) and DIEA (6.00 eq) |
| 35 | Fmoc-Ser(tBu)-OH (3.00 eq) | HATU (2.85 eq) and DIEA (6.00 eq) |
| 36 | Fmoc-Ile-OH (3.00 eq) | HATU (2.85 eq) and DIEA (6.00 eq) |
| 37 | Fmoc-Phe-OH (3.00 eq) | HATU (2.85 eq) and DIEA (6.00 eq) |
| 38 | Fmoc-Thr(tBu)-OH (3.00 eq) | HATU (2.85 eq) and DIEA (6.00 eq) |
| 39 | Fmoc-Gly-OH (3.00 eq) | HATU (2.85 eq) and DIEA (6.00 eq) |
| 40 | Fmoc-Glu(OtBu)-OH (3.00 eq) | HATU (2.85 eq) and DIEA (6.00 eq) |
| 41 | Fmoc-Aib-OH (3.00 eq) | HATU (2.85 eq) and DIEA (6.00 eq) |
| 42 | Boc-Tyr(tBu)-OH (3.00 eq) | HATU (2.85 eq) and DIEA (6.00 eq) |

5. After the coupling of tert-butoxycarbonyl tert-butyl carbonate, 3% H₂N—NH₂/DMF was added and allowed to react for 30 min. The reaction was repeated, and then drained and washed with DMF (20.0 mL×5).
6. Steps 2 and 3 were repeated using the reagents in Table 2 for the coupling of the remaining amino acids (43-45):

TABLE 2

| # | Materials | Coupling reagents |
|---|---|---|
| 43 | Fmoc-Lys(Boc)-OH | HATU (2.85 eq) and |
| | (3.00 eq) | DIEA (6.00 eq) |
| 44 | Fmoc-Glu-OtBu | HATU (2.85 eq) and |
| | (3.00 eq)* | DIEA (6.00 eq) |
| 45 | C₂₀Diacid(tBu) | HATU (2.85 eq) and |
| | (3.00 eq) | DIEA (6.00 eq) |

These groups 43-45 are residues 47 and Z in the structural definition in Figure 1 and in the claims.
*As the protected glutamic acid reagent 44 has its C-1 acid group protected with tBu, it reacts at its C-5 acid with the lysine 43. It is the Fmoc-Glu-OtBu reagent that provides the glutamic acid residue portion in the Z part of the compound when Z is (i):

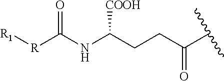

Peptide cleavage and purification:
7. The resin was washed with MeOH (20.0 mL×2) and dried under vacuum to provide 3 g peptide resin. Then 30 mL of cleavage buffer (92.5% TFA/2.5% 3-Mpr/2.5% TIS/2.5% H₂O) was added to the flask containing the side-chain-protected peptide resin at 20° C. and the mixture was stirred for 2 h. The peptide was precipitated with cold isopropyl ether (300 mL) and centrifuged (2 min at 3000 rpm). The peptide precipitate was washed with isopropyl ether twice more (300 mL). The crude peptide was dried under vacuum for 2 h, and the identity was confirmed by LCMS.
8. The residue was purified by prep-HPLC (TFA conditions; 30° C., A: 0.075% TFA/H₂O, B: CH₃CN) to give the title compound (244.5 mg, 43.2 μmol, 14.4% yield, 98.3% purity, TFA) as a white solid, the identity of which was confirmed by LCMS.

Equivalent methods were employed for all of the other peptides described herein. The sequences and other structural features of the exemplified peptides are shown in FIG. 1. In the figure, the Lys* residue is substituted on its ε amino group with a group Z-Xaa44-Xaa45-Xaa46-Xaa47—(blank in the column in question means that the residue is absent), and Z is a group having the structure:

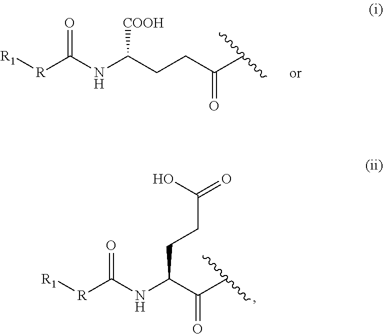

wherein R is a C₈-C₂₈ alkylene or alkenylene group; and
R₁ is CO₂H.

The number of carbon atoms in the alkylene or alkenylene group of R (n, n=8-28) is indicated in the column headed “n (R=Cn)” in FIG. 1. In the examples shown, n is 14, 16 or 18.

Certain of the example compounds in FIG. 1 are duplicate preparations of compounds listed elsewhere in the Figure and they are indicated as such as “dupl” in the column headed ‘Notes’.

### Receptor Potency of Peptides at the Human GIP Receptor, Overexpressed in HEK Cells

Biological activity was assessed by potency of peptides to stimulate CAMP production in Human Embryonic Kidney (HEK) cell lines overexpressing the SNAP-tagged human GIP receptor. Expression of the receptor was induced by the addition of tetracycline to cell culture media 24 h prior to use. Cells were plated at a density of 8×10⁻⁵ cells/mL in serum-free media, into 96-well half area plates, on the day of the assay. A commercial CAMP kit (Cisbio) was used to quantify CAMP in the cell via HTRF (Homogeneous Time-Resolved Fluorescence) technology after 30 mins of peptide stimulation and a further 1 h lysis. Plates were read on a SpectraMax i3x Multi-Mode Detection Platform plate reader and concentration response curves drawn with Graph Pad Prism 8.0 (or higher). EC₅₀ values were generated for each peptide and compared to the controls for the day.

### In Vivo Efficacy Study: Single Dose Feeding Studies in Male Mice

Male mice (Charles River Ltd, Margate, UK) were used for animal experiments. Individually housed mice in IVC cages were randomised into treatment groups, with stratification by body weight. Mice were fasted overnight prior to injection. All peptide solutions were prepared freshly, immediately prior to administration. The control animals were dosed with 5% v/v water and 95% NaCl (0.9% w/v). Treatment groups received a single subcutaneous injection of 10⁻⁴⁵ nmol/kg body weight GIP analogue in combination with 1-1.5 nmol/kg body weight GLP-1r agonist G7097, prepared in water for injection. At the time of injection (early light phase; 0900-1000), body weight was measured in grams (g) and each mouse was re-fed a known amount of food. Subsequent remaining food and body weight measurements were taken up to 72 h after dosing. Animals were given free access to water during the study period.

The results for Example compounds 1 to 110 are shown in FIG. 1. Mice were fasted overnight and were dosed in the early light phase, then given a known amount of food. Mice were dosed with a single subcutaneous injection, combining a sub-therapeutic dose of a GLP-1r agonist G7097 (0.5-1 nmol/kg) and an Example compound (4.5-30 nmol/kg). In the Table in FIG. 1, column ‘n’ shows the number of times the compound in question was tested. Each test generally involved the compound being given to a group of 5 animals. The compounds were assessed for their propensity to inhibit food intake and their ability to bring about body weight change. The summation of their actions is reported as a single value, called “x”, representing potency, in FIG. 1. To score “potency”, average food intake difference, measured in grams, compared to vehicle controls, was measured at at least two of the following time points: 24, 48 and 72 h post dose multiplied by the average body weight difference, measured in grams, compared to vehicle controls measured at at least two of the following time points: 24, 48 and 72 h post dose. The higher the number, the more potent the analogue.

### PK Measurements for Half-Life Assessment

Göttingen minipigs received a single subcutaneous injection of the test compound at the start of study (0 hours). Repeated blood samples were taken from the pig over the proceeding 216 hours and plasma separated and stored at −20 to −80° C. Plasma concentration of the test compound was quantified using mass spectrum analysis. Time taken from tₘₐₓ to half of Cₘₐₓ was calculated and represents the half-life (t_{1/2}) of the compound. Typically, the presented t_{1/2} is the mean of two separate animals.

### Results

FIG. 1 is a table providing amino acid sequences and other structural information for the example compounds of the invention. For example, the amino acid sequence of example compound no. 1 is as follows [SEQ ID NO: 12]:

Tyr Ala Glu Gly Thr Phe Ile Ser Asp Tyr Ser Ile
Ala Met Asp Lys Ile His Gln Gln Asp Phe Val Asn
Trp Leu Leu Ala Gln Lys Gly Lys Lys Asn Asp Trp
Lys His Asn Ile Thr Gln Lys*

And the Lys* residue at position 43 carries on its ε-amino group the group

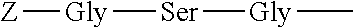

where Z is a group of formula:

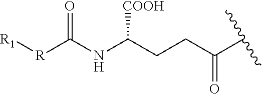

wherein R is a C₁₈ straight chain alkylene group; and
R₁ is CO₂H.

The figure also summarises the results of the in vivo feeding efficacy studies with the example peptides of the invention as discussed above.

To the right of the columns showing the feeding scores, the figure shows receptor potency data for example peptides at the GIP receptor, overexpressed in Human

Embryonic Kidney (HEK) cells. Biological activity was assessed by the potency of peptides to stimulate cAMP production in the HEK cells as described above. As can be seen from the Figure, mice which were fasted overnight and which were administered example peptides of the invention in combination with a GLP-1r agonist G7097, achieved reduced weight gain or achieved weight loss compared with mice which were administered G7097 alone or vehicle control animals. This supports that the compounds of the invention are particularly effective at improving metabolism, and that they find use in the therapy of disorders such as obesity.

### Half-Life:

The half-life of a selection of compounds of the invention was measured. The results, in hours, are shown in the column labelled t_{1/2} at the far right of the table in FIG. 1.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A compound of formula:
[SEQ ID NO: 1]
Xaa1-Xaa2-Xaa3-Gly4-Thr5-Phe6-Xaa7-Ser8-Asp9-Xaa10-Ser11-Xaa12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-Xaa30-Xaa31-Xaa32-Xaa33-Xaa34-Xaa35-
Xaa36-Xaa37-Xaa38-Xaa39-Xaa40-Xaa41-Xaa42-Xaa43
wherein:
Xaa1 is DTyr, His, Phe or Tyr, or absent;
Xaa2 is AIB or Ala, or absent;
Xaa3 is Asp, Gln, Glu or His;
Xaa7 is IIe, Thr or Ser;
Xaa10 is Tyr or His;
Xaa12 is IIe or Lys;
Xaa13 is AIB, Ala, Gln, Glu, Gly, His, Pro, Ser, Thr, Tyr, Val, Asp or Asn;
Xaa14 is Leu, Met or NLeu;
Xaa17 is Gln or IIe;
Xaa18 is Ala, Arg or His;
Xaa19 is Ala or Gln;
Xaa20 is AIB, Gln, His, or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn;
Xaa29 is Gln, Gly or Thr;
Xaa30 is Gly, Lys or Lys*, or absent;
Xaa31 is Arg, Gly or Pro, or absent;
Xaa32 is Asn, Lys or Lys*, or absent;
Xaa33 is Lys or Lys*, or absent;
Xaa34 is Asn or Lys*, or absent;
Xaa35 is Asp or absent;
Xaa36 is Trp or Lys*, or absent;
Xaa37 is Lys or Lys*, or absent;
Xaa38 is His or absent;
Xaa39 is Asn or Lys*, or absent;
Xaa40 is IIe, Leu or Lys*, or absent;
Xaa41 is Thr, or absent;
Xaa42 is Gln, or absent;
Xaa43 is Lys* or absent;
wherein Lys* is Lys substituted at its ε-amino group with a group of the formula: 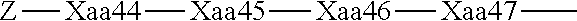
wherein:
Xaa44 is Gly, Ser, Thr or absent;
Xaa45 is Gly, Ser or absent;
Xaa46 is Asn, Gln, Gly, Ser, Thr or absent;
Xaa47 is Asn, Gln, Glu, Gly, His, Lys, Pro, Ser, Thr or absent;
and Z is a group of the formula: 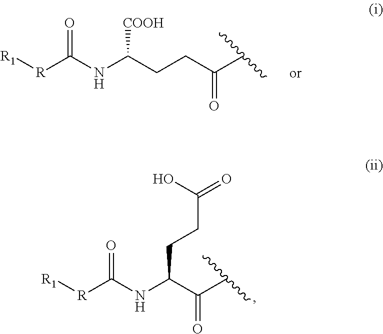
wherein R is a C₈-C₂₈ alkylene or alkenylene chain and R¹ is —CO₂H;
and wherein the compound of formula (I) or formula (II) contains one Lys* residue;
or a derivative of the compound; or a salt or solvate of the compound or the derivative.

2. A compound, derivative, salt or solvate as claimed in claim 1, wherein the compound is a compound of formula (I) or formula (II): 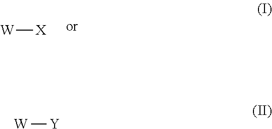
wherein
W is an amino acid sequence:
[SEQ ID NO: 7]
Xaa1-Xaa2-Xaa3-Gly4-Thr5-Phe6-Xaa7-Ser8-Asp9-Xaa10-Ser11-Xaa12-Xaa13-
Xaa14-Asp15-Lys16-Xaa17-Xaa18-Xaa19-Xaa20-Xaa21-Phe22-Val23-Xaa24-
Xaa25-Leu26-Xaa27-Xaa28-Xaa29-
wherein:
Xaa1 is DTyr, His, Phe or Tyr, or absent;
Xaa2 is AIB or Ala, or absent;
Xaa3 is Asp, Gln, Glu or His;
Xaa7 is IIe, Thr or Ser;
Xaa10 is Tyr or His;
Xaa12 is IIe or Lys;
Xaa13 is AIB, Ala, Gln, Glu, Gly, His, Pro, Ser, Thr, Tyr, Val, Asp or Asn;
Xaa14 is Leu, Met or NLeu;
Xaa17 is Gln or IIe;
Xaa18 is Ala, Arg or His;
Xaa19 is Ala or Gln;
Xaa20 is AIB, Gln, His or Lys;
Xaa21 is Ala, Asp or Glu;
Xaa24 is Asn, Gln or Glu;
Xaa25 is Arg, His, Trp or Tyr;
Xaa27 is IIe or Leu;
Xaa28 is Ala or Asn; and
Xaa29 is Gln, Gly or Thr;
X, when present, is an amino acid sequence:
[SEQ ID NO: 8]
-Xaa30-Xaa31-Xaa32-Xaa33-Asn34-Asp35-Xaa36-Xaa37-His38-Xaa39-Xaa40-
Thr41-Gln42-Xaa43
wherein:
Xaa30 is Gly, Lys or Lys*;
Xaa31 is Gly or Pro;
Xaa32 is Lys or Lys*;
Xaa33 is Lys or Lys*;
Xaa36 is Trp or Lys*;
Xaa37 is Lys or Lys*;
Xaa39 is Asn or Lys*;
Xaa40 is IIe, Leu or Lys*; and
Xaa43 is Lys* or absent;
Y, when present, is an amino acid sequence: 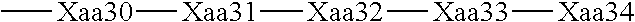
wherein:
Xaa30 is Gly or Lys, or absent;
Xaa31 is Arg, Gly or Pro, or absent;
Xaa32 is Asn or Lys, or absent;
Xaa33 is Lys, Lys* or absent; and
Xaa34 is Lys* or absent;
wherein Lys* is Lys substituted at its ε-amino group with a group of the formula: 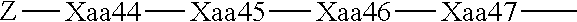
wherein:
Xaa44 is Gly, Ser or Thr, or absent;
Xaa45 is Gly or Ser, or absent;
Xaa46 is Asn, Gln, Gly, Ser or Thr, or absent;
Xaa47 is Asn, Gln, Glu, Gly, His, Lys, Pro, Ser or Thr, or absent;
and Z is a group of the formula: 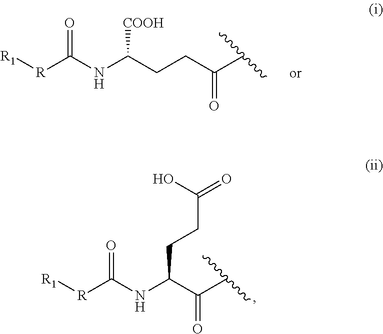
wherein R is a C₈-C₂₈ alkylene or alkenylene chain and R¹ is —CO₂H;
and wherein the compound of formula (I) or formula (II) contains one Lys* residue;
or a derivative of the compound; or a salt or solvate of the compound or the derivative.

3. A compound, derivative, salt or solvate as claimed in claim 2 wherein:
Xaa1 is His, Phe or Tyr;
Xaa2 is AIB;
Xaa3 is Gln, Glu or His;
Xaa7 is IIe or Ser;
Xaa10 is Tyr or His;
Xaa12 is IIe or Lys;
Xaa13 is Ala, Gln, Thr, Val, Asp or Asn;
Xaa14 is Leu or Met;
Xaa17 is Gln or IIe;
Xaa18 is Ala or His;
Xaa19 is Ala or Gln;
Xaa20 is AIB, Gln, His or Lys;
Xaa21 is Asp or Glu;
Xaa24 is Asn, Gln or Glu;
Xaa25 is Trp;
Xaa27 is Leu;
Xaa28 is Ala or Asn; and
Xaa29 is Gln, Gly or Thr.

4. A compound, derivative, salt or solvate as claimed in claim 3 wherein:
Xaa1 is Tyr;
Xaa2 is AIB;
Xaa3 is Glu or Gln;
Xaa7 is IIe or Ser;
Xaa10 is Tyr or His;
Xaa12 is IIe or Lys;
Xaa13 is Ala, Asp or Asn;
Xaa14 is Leu;
Xaa17 is IIe;
Xaa18 is His;
Xaa19 is Gln;
Xaa20 is Gln or Lys;
Xaa21 is Asp;
Xaa24 is Asn or Gln;
Xaa25 is Trp;
Xaa27 is Leu;
Xaa28 is Ala; and
Xaa29 is Gln or Gly.

5. A compound, derivative, salt or solvate as claimed in claim 4 wherein:
Xaa3 is Glu;
Xaa20 is Gln;
Xaa24 is Asn; and
Xaa29 is Gln.

6. A compound, derivative, salt or solvate as claimed in any one of claims 2 to 5, wherein the compound is a compound of formula (I).

7. A compound, derivative, salt or solvate as claimed in claim 6, wherein:
Xaa30 is Lys or Lys*;
Xaa31 is Gly;
Xaa32 is Lys or Lys*;
Xaa33 is Lys or Lys*;
Xaa36 is Trp or Lys*;
Xaa37 is Lys or Lys*;
Xaa39 is Asn or Lys*;
Xaa40 is IIe or Lys*; and
Xaa43 is Lys* or absent.

8. A compound, derivative, salt or solvate as claimed in claim 7, wherein:
Xaa30 is Lys;
Xaa32 is Lys*;
Xaa33 is Lys;
Xaa36 is Trp;
Xaa37 is Lys;
Xaa39 is Asn;
Xaa40 is IIe; and
Xaa43 is absent.

9. A compound, derivative, salt or solvate as claimed in any one of claims 2 to 5, wherein the compound is a compound of formula (II).

10. A compound, derivative, salt or solvate as claimed in claim 9, wherein:
Xaa30 is Lys or Gly;
Xaa31 is Gly or Pro;
Xaa32 is Lys or absent;
Xaa33 is Lys, Lys* or absent; and
Xaa34 is Lys* or absent.

11. A compound, derivative, salt or solvate as claimed in claim 10, wherein:
Xaa30 is Lys;
Xaa31 is Gly;
Xaa32 is Lys or absent;
Xaa33 is Lys, Lys* or absent; and
Xaa34 is Lys* or absent.

12. A compound, derivative, salt or solvate as claimed in claim 1, which has an amino acid sequence corresponding to any one of the amino acid sequences listed in the Table of FIG. 1.

13. A derivative of a compound as claimed in any of claims 1 to 12, or a salt or solvate of such a derivative, which comprises one or more derivatisations selected from amidation, glycosylation, carbamylation, acylation, sulfation, phosphorylation, cyclization, lipidization, pegylation and fusion to another peptide or protein to form a fusion protein.

14. A compound, derivative, salt or solvate as claimed in any of claims 1 to 13 together with a further therapeutic agent, for simultaneous, sequential or separate administration.

15. A compound, derivative, salt or solvate as claimed in claim 14, wherein the further therapeutic agent is a GLP-1 receptor agonist or an analogue of amylin.

16. A composition comprising a compound, derivative, salt or solvate as claimed in any of claims 1 to 15 together with a pharmaceutically acceptable carrier.

17. A composition as claimed in claim 16, present in a syringe or other administration device for subcutaneous administration to humans.

18. A compound, derivative, salt or solvate as claimed in any of claims 1 to 15, or a composition as claimed in claim 16 or claim 17 for use as a medicament.

19. A method of treating or preventing a disease or disorder or other non-desired physiological state in a subject comprising administration of a therapeutically effective amount of a compound, derivative, salt or solvate as claimed in any of claims 1 to 15, or a composition as claimed in claim 16 or claim 17.

20. A compound, derivative, salt or solvate as claimed in any of claims 1 to 15, or a composition as claimed in claim 16 or claim 17, for use in the prevention or treatment of diabetes, obesity, heart disease, stroke and non-alcoholic fatty liver disease, improving insulin release in a subject, improving carbohydrate metabolism in a subject, improving the lipid profile of a subject, reducing appetite, reducing food intake, reducing calorie intake, improving carbohydrate tolerance in a subject, and/or for use as a cytoprotective agent.

21. A compound, derivative, salt or solvate or composition for use as a cytoprotective agent as claimed in claim 20, wherein the compound, derivative, salt or composition is for use in the prevention or treatment of neurodegeneration, providing neuroprotection and/or providing cardiac protection.

22. A compound, derivative, salt or solvate or composition for use as a cytoprotective agent as claimed in claim 21, wherein the compound, derivative, salt or composition is for providing cardiac protection in a subject following a myocardial infarction.

23. A compound, derivative, salt or solvate or composition for use as a cytoprotective agent as claimed in claim 22, wherein the compound, derivative, salt or composition is for providing neuroprotection in a subject having or diagnosed as being at risk of a chronic neurodegenerative disease.

24. A compound, derivative, salt or solvate or composition for use as claimed in claim 23, wherein the chronic neurodegenerative disease is selected from the group consisting of Alzheimer's disease, Parkinson's disease, Gehrig's disease (Amyotrophic Lateral Sclerosis), Huntington's disease, Multiple Sclerosis, other demyelination related disorders, senile dementia, subcortical dementia, arteriosclerotic dementia, AIDS-associated dementia, other dementias, cerebral vasculitis, epilepsy, Tourette's syndrome, Guillain Barre Syndrome, Wilson's disease, Pick's disease, neuroinflammatory disorders, encephalitis, encephalomyelitis, meningitis, other central nervous system infections, prion diseases, cerebellar ataxias, cerebellar degeneration, spinocerebellar degeneration syndromes, Friedrich's ataxia, ataxia teangiectasia, spinal dysmyotrophy, progressive supranuclear palsy, dystonia, muscle spasticity, tremor, retinitis pigmentosa, striatonigral degeneration, mitochondrial encephalomyopathies and neuronal ceroid lipofuscinosis.

25. A method of treating or preventing diabetes, obesity, heart disease, stroke or non-alcoholic fatty liver disease in a subject, improving insulin release in a subject, improving carbohydrate metabolism in a subject, improving the lipid profile of a subject, improving carbohydrate tolerance in a subject, reducing appetite, reducing food intake, reducing calorie intake, and/or providing cytoprotection in a subject, comprising administration of a therapeutically effective amount of a compound, derivative, salt or solvate as claimed in any one of claims 1 to 15, or a composition as claimed in claim 16 or claim 17.

26. Use of a compound, derivative, salt or solvate as claimed in any one of claims 1 to 15 for the manufacture of a medicament for the prevention or treatment of diabetes, obesity, heart disease, stroke and non-alcoholic fatty liver disease, improving insulin release in a subject, improving carbohydrate metabolism in a subject, improving the lipid profile of a subject, improving carbohydrate tolerance in a subject, reducing appetite, reducing food intake, reducing calorie intake, and/or for use as a cytoprotective agent.

27. A method of causing weight loss or preventing weight gain in a subject for cosmetic purposes comprising administration of an effective amount of a compound, derivative, salt or solvate as claimed in any one of claims 1 to 15, or a composition as claimed in claim 16 or claim 17.
